(19) United States
(12) Patent Application Publication
(10) Pub. No.: US 2006/0127409 A1
(43) Pub. Date: Jun. 15, 2006

(54) Bcr-abl vaccines and methods of use thereof

(76) Inventors: David A. Scheinberg, New York, NY (US); Javier Pinilla-Ibarz, New York, NY (US)

Correspondence Address: PEARL COHEN ZEDEK, LLP, 1500 BROADWAY 12TH FLOOR, NEW YORK, NY, 10036, US

(21) Appl. No.: 11/250,607

(22) Filed: Oct. 17, 2005

Related U.S. Application Data
(63) Continuation-in-part of application No. 10/999,425, filed on Nov. 30, 2004.
(60) Provisional application No. 60/525,955, filed on Dec. 1, 2003.

(51) Int. Cl. A61K 39/00; A61K 38/17

(52) U.S. Cl. 424/185.1; 514/12

(57) ABSTRACT

This invention provides vaccine comprising immunogenic bcr-abl peptides and methods of treating, inhibiting the progression of, reducing the incidence of, and breaking a T cell tolerance of a subject to a bcr-abl-associated cancer.

### CROSS REFERENCE TO RELATED APPLICATIONS

This application is a continuation-in-part of U.S. application Ser. No. 10/999,425, filed Nov. 30, 2004, which claims priority of U.S. Provisional Application Ser. No. 60/525,955, filed Dec. 1, 2003. These applications are hereby incorporated in their entirety by reference herein.

### STATEMENT REGARDING FEDERALLY SPONSORED RESEARCH OR DEVELOPMENT

This invention described herein was supported in part by a grant from The National Institutes of Health (Grant No. CA 23766). The U.S. Government may have certain rights in this invention.

### FIELD OF THE INVENTION

This invention provides vaccine comprising immunogenic bcr-abl peptides and methods of treating, inhibiting the progression of, reducing the incidence of, and breaking a T cell tolerance of a subject to a bcr-abl-associated cancer.

### BACKGROUND OF THE INVENTION

Leukemias, including chronic myelogenous leukemia (CML), acute myelogenous leukemia (AML) and acute lymphocytic leukemia (ALL) are pluripotent stem cell disorders, which may be characterized by the presence of the Philadelphia chromosome (Ph). Because of the unique features, these cancers present a unique opportunity to develop therapeutic strategies using vaccination against a truly tumor specific antigen that is also the oncogenic protein required for neoplasia.

The chimeric fusion proteins are potential antigens for two reasons. The proteins are uniquely expressed in the leukemic cells in which the junctional regions contain a sequence of amino acids that is not expressed on any normal protein. In addition, as a result of the codon split on the fused message, a new amino acid (lysine in b3a2) and a conserved one (glutamic acid in b2a2) are present at the exact fusion point in each of the proteins. Therefore, the unique amino acid sequences encompassing the b3a2 and b2a2 breakpoint region can be considered truly tumor specific antigens. Despite the intracellular location of these proteins, short peptides produced by cellular processing of the products of the fusion proteins can be presented on the cell surface within the cleft of human leukocyte antigen (HLA) molecules, and in this form, may be recognized by T cells.

Tumor specific, bcr-abl derived multivalent vaccine can be safely administered to patients with chronic phase CML; the vaccine reliably elicits a bcr-abl peptide specific CD4 immune response, as measured by DTH in vivo, CD4⁺ T cell proliferation ex vivo and gamma interferon secretion in a ELISPOT assay. CD8 responses in A0201 patients, however, were undetectable, and only weak responses in HLA A0301 patients using a sensitive gamma interferon ELISPOT assay were found. For stimulation of responses the strength of CD8 responses depends upon the binding affinity of the target peptide to class I MHC molecules, the peptide-HLA complex stability, and the avidity of the T cell receptor binding for the peptide complex. Killing of native CML cells also requires adequate processing and presentation of the natural antigen. Therefore the lack of reproducible CD8 responses may reflect the biochemistry of the class I peptide-HLA interaction, which resulted in their weak immunogenicity to cytotoxic CD8 cells

Thus, there remains a need to design peptides that are more immunogenic and that produce a robust CTL, response. Ideally, such peptides should generate an immune response that not only recognizes the immunizing epitopes, but also that cross reacts with the original native peptides, producing a heteroclitic response, which as yet, is lacking.

### SUMMARY OF THE INVENTION

This invention provides vaccine comprising immunogenic bcr-abl peptides and methods of treating, inhibiting the progression of, reducing the incidence of, and breaking a T cell tolerance of a subject to a bcr-abl-associated cancer.

In one embodiment, the present invention provides a bcr-abl vaccine comprising an unmutated bcr-abl peptide and a mutant bcr-abl peptide. In another embodiment, the bcr-abl vaccine further comprises an adjuvant. The unmutated bcr-abl peptide corresponds, in one embodiment, to a first bcr-abl breakpoint fragment. In another embodiment the mutant bcr-abl peptide is a human leukocyte antigen (HLA) class I-binding peptide, and corresponds to a second bcr-abl breakpoint fragment with a mutation in an anchor residue of the second bcr-abl breakpoint fragment.

In another embodiment, the mutant bcr-abl peptide comprises a HLA class I-binding peptide, wherein the HLA class I-binding peptide corresponds to a second bcr-abl breakpoint fragment with a mutation in an anchor residue of the second bcr-abl breakpoint fragment.

In another embodiment, the present invention provides a method of treating a subject with a bcr-abl-associated cancer, the method comprising administering to the subject a bcr-abl vaccine of the present invention, thereby treating a subject with a bcr-abl-associated cancer.

In another embodiment, the present invention provides a method of reducing the incidence of a bcr-abl-associated cancer, or its relapse, in a subject, the method comprising administering to the subject a bcr-abl vaccine of the present invention, thereby reducing an incidence of a bcr-abl-associated cancer, or its relapse, in a subject.

In another embodiment, the present invention provides a method of breaking a T cell tolerance of a subject to a bcr-abl-associated cancer, the method comprising administering to the subject a bcr-abl vaccine of the present invention, thereby breaking a T cell tolerance to a bcr-abl-associated cancer.

In another embodiment, the present invention provides a bcr-abl vaccine comprising peptides having the sequences VHSIPLTINKEEALQRPVASDFE (SEQ ID No: 17) and YLINKEEAL (SEQ ID No: 14). In another embodiment, the bcr-abl vaccine further comprises an adjuvant.

In another embodiment, the present invention provides a bcr-abl vaccine comprising peptides having the sequences IVHSATGFKQSSKALQRPVASDFE (SEQ ID No: 18), KQSSKALQR (SEQ ID No: 3), GFKQSSKAL (SEQ ID No: 19), KLLQRPVAV (SEQ ID No: 7), and YLKALQRPV (SEQ ID No: 2) In another embodiment, the bcr-abl vaccine further comprises an adjuvant.

### BRIEF DESCRIPTION OF THE DRAWINGS

FIG. 1. A. Specific proliferation of human T cells in response to stimulation with b3a2-CML peptide (SEQ ID No: 18) After two sets of stimulations with b3a2-CML pulsed autologous PBMC, T cells were incubated with irradiated (first bar in each series) or paraformaldehyde-fixed (second bar in each series; negative control) autologous PBMC that were either not peptide-pulsed (T cells+APC); pulsed with b3a2-CML peptide (T cells+APC+b3a2 CML); or pulsed with a control peptide (T cells+APC+CDR2). (In groups in which the separation between the first and second bar in a series is unclear, a dotted line is provided to show the demarcation) Specific proliferation measured by ³H-thymidine incorporation after 72 hours of culture is depicted. The data show specific proliferation of T cells incubated with b3a2-CML peptide-pulsed autologous PBMC as antigen presenting cells. No proliferation was observed when no peptide was added to the APC or APC where pulsed with the control peptide. B. IFN-γ production in response to b2a2 long peptide (SEQ ID No: 17). T cells: no APC were added. WT1-DR, b3a2, b2a2-: APC+the indicated peptide were added. APC: APC alone were added.

FIG. 2 depicts results of a T2 stabilization assay using peptides derived from b3a2 translocation (left panel) and b2a2 translocations (right panel). Peptide sequences are delineated in Table 1. The fluorescence index is the value obtained for the ratio between median fluorescence obtained with the indicated peptide divided by background fluorescence. The X-axis represents different peptide concentrations. “n” denotes native sequences from b3a2. p210Cn, p210Dn, CMLA2, and CMLA3 are native b3a2 sequences; b2a2A is the native sequence for b2a2.

FIG. 3 depicts gamma interferon (IFN) production detected by ELISPOT of CD8⁺ T cells from a healthy HLA A0201 donor following two in vitro stimulations with the peptides p210 C and F. After stimulation, CD8⁺ cells were challenged with the following: T2 (APC), or T2 pulsed with tested peptide (p210C or p210F), corresponding native peptide, or negative control peptide, as indicated.

FIG. 4 depicts secretion of gamma IFN detected by ELISPOT of CD8⁺ T cells from an HLA A0201, chronic phase CML patient following two in vitro stimulations with p210C. T cells were challenged with the following: media, APC T2, or T2 pulsed with p210C, corresponding native peptide, or negative control peptide. Empty bars: CD8⁺ cells plus media. Dot bars: CD8⁺ plus APC T2. Diagonal bars: CD8⁺ plus T2 pulsed with p210C. Black bars: CD8⁺ plus T2 pulsed with corresponding native peptide p210Cn. Grey bars: CD8⁺ plus T2 pulsed with irrelevant control peptide.

FIG. 5 depicts production of gamma IFN detected by ELISPOT of CD3⁺ cells of two healthy HLA A0201 donors after two in vitro stimulations with the indicated bcr-abl peptides. T cells were challenged with the following: media, APC T2, or T2 pulsed with test peptide (b2a2 A3, A4 or A5); corresponding native peptide, or negative control peptide. Dot bars: CD8⁺ plus APC T2. diagonal bars: CD8⁺ plus T2 pulsed with tested peptide (b2a2 A3, A4 or A5). black bars: CD8⁺ plus T2 pulsed with native peptide (cross reactivity). Grey bars: CD8⁺ plus T2 pulsed with irrelevant control peptide.

FIG. 6 depicts results of a cytotoxicity assay with T cells isolated from a healthy HLA A0201 donor following three in vitro stimulations with p210F. Target cells used were T2 cell lines pulsed with the indicated peptides The Y-axis reflects the percent cytotoxicity, and the X-axis reflects the varied T cell/target ratio. Open squares: T2 with no peptide. Open diamonds: T2 pulsed with p210F. Open circles: T2 pulsed with CMLA2. Open triangles: T2 pulsed with irrelevant control peptide.

FIG. 7 depicts results of two cytotoxicity assays with T cells isolated from a healthy HLA A0201 donor following five in vitro stimulations with b2a2 A3 peptide. Target cells used were T2 cell line pulsed with the indicated peptides. Y-axis reflects the percent cytotoxicity, and the X-axis reflects the different T cell/target ratio. Open squares: T2 with no peptide. Open diamond: T2 pulsed with b2a2 A3 peptide. Open circles: T2 pulsed with negative control peptide.

FIG. 8. CD4⁺ T cell responses to administration of b2a2 long peptide. A. baseline. B. 2 weeks after fifth vaccination. “b2a2 longbulk”=mixture of long and short b2a2 peptide. b2a2L=b2a2 long peptide. “Ras”=ras protein control. “Bulk”=mixture of negative controls

### DETAILED EMBODIMENTS OF THE INVENTION

This invention provides vaccine comprising immunogenic bcr-abl peptides and methods of treating, inhibiting the progression of, reducing the incidence of, and breaking a T cell tolerance of a subject to a bcr-abl-associated cancer.

As provided herein, bcr-abl breakpoint-derived peptides that stimulated HLA class II molecules were identified. As provided herein, vaccines comprising both mutated and wild-type bcr-abl breakpoint-derived peptides are particularly efficacious in eliciting anti-bcr-abl immune responses and in treating and preventing bcr-abl associated cancers (Examples 7-9).

In one embodiment, the present invention provides a bcr-abl vaccine comprising an unmutated bcr-abl peptide and a mutant bcr-abl peptide. In another embodiment, the bcr-abl vaccine further comprises an adjuvant. The unmutated bcr-abl peptide corresponds, in one embodiment, to a first bcr-abl breakpoint fragment. The mutant bcr-abl peptide is, in another embodiment, a human leukocyte antigen (HLA) class I-binding peptide, and corresponds to a second bcr-abl breakpoint fragment with a mutation in an anchor residue of the second bcr-abl breakpoint fragment. Bcr-abl vaccines of the present invention elicit, in another embodiment, immune responses against cells presenting bcr-abl breakpoint fragments corresponding to the bcr-abl peptides in the vaccine.

In another embodiment, the mutant bcr-abl peptide comprises a HLA class I-binding peptide, wherein the HLA class I-binding peptide corresponds to a second bcr-abl breakpoint fragment with a mutation in an anchor residue of the second bcr-abl breakpoint fragment.

For example, in one embodiment of the above vaccine, the unmutated bcr-abl peptide has the sequence IVHSATGFKQSSKALQRPVASDFE (SEQ ID No: 18) and the mutant bcr-abl peptide has the sequence KLLQRPVAV (SEQ ID No: 7). In this embodiment, the sequence of the first bcr-abl breakpoint fragment is IVHSATGFKQSSKALQRPVASDFE, identical to that of the unmutated bcr-abl peptide. The sequence of the HLA class I-binding peptide in this embodiment is KLLQRPVAV, identical to that of the mutant bcr-abl peptide. The sequence of the second bcr-abl breakpoint fragment in this embodiment is KALQRPVAS (SEQ ID No: 6). The mutant bcr-abl peptide of this embodiment was generated from the second bcr-abl breakpoint fragment by mutation of residues 2 and 9 to leucine and valine, respectively.

Bcr-abl is a fusion gene associated, inter alia, with chronic myelogenous leukemia (CML), and results from a translocation of the c-abl oncogene from chromosome 9 to the specific breakpoint cluster region (bcr) of the BCR gene on chromosome 22. The t(9;22) (q34; q11) translocation is present in more than 95% of patients with CML. The translocation of the c-abl to the breakpoint cluster region (bcr) forms bcr-abl, which, in one embodiment, is a 210 kD chimeric protein with abnormal tyrosine kinase activity.

In another embodiment, bcr-abl is typically expressed only by leukemia cells. In another embodiment, bcr-abl can stimulate the growth of hematopoietic progenitor cells and contribute to pathogenesis of leukemia. In other embodiments, the bcr breakpoint is between exons 2 and 3 or exons 3 and 4. In another embodiment, the bcr-abl reading frames are fused in frame, and the translocated mRNA encodes a functional 210 kD chimeric protein consisting of 1,004 c-abl encoded amino acids plus either 902 or 927 bcr encoded amino acids—both of which are enzymatically active as protein kinases.

In another embodiment, the bcr-abl protein of methods and compositions of the present invention results from a translocation associated with acute lymphoblastic leukemia (ALL), wherein c-abl is translocated to chromosome 22 but to a different region of the bcr gene, denoted BCRI, which results in the expression of a p185-190^{bcr-abl} chimeric protein kinase. p185-190^{bcr-abl} is expressed in approximately 10% of children and 25% of adults with ALL.

The bcr-abl protein of methods and compositions of the present invention can be any bcr-abl protein known in the art. In another embodiment, the bcr-abl protein has the sequence set forth in GenBank Accession #. In other embodiments, the bcr-abl protein has or comprises one of the sequences set forth in one of the following sequence entries: X02596, NM_{—}004327, X02596, U07000, Y00661, X06418, NM_{—}005157, NM_{—}007313, U07563, M15025, BAB62851, AAL05889, AAL99544, CAA10377, CAA10376, AAD04633, M14752, M14753, AAA35592, AAA35594, AAA87617, AAA88013, 1314255A, AAF61858, AAA35596, AAF89176, AAD04633, In another embodiment, the bcr-abl protein has any other bcr-abl sequence known in the art.

In another embodiment, the bcr-abl protein is derived from the translated product of a bcr-abl translocation event that is associated with a neoplasm. In one embodiment, the neoplasm is a leukemia, which is, in other embodiments, a CML, AML, or ALL.

Each of the above bcr-abl proteins or types thereof represents a separate embodiment of the present invention.

Bcr-abl peptides of methods and compositions of the present invention are, in another embodiment, derived from junctional sequences of one of the above bcr-abl proteins. “Junctional sequences” (“breakpoint sequences”) refers, in one embodiment, to sequences that span the fusion point of bcr-abl or another protein that arises from a translocation. Peptides derived from bcr-abl breakpoint sequences that naturally occur in cancer cells are referred to, in another embodiment, as “bcr-abl breakpoint fragments.”

For purposes of readability, the bcr-abl peptides used in vaccines of the present invention (e.g. the unmutated bcr-abl peptide and mutant bcr-abl peptide in the above vaccine) are referred to below as “bcr-abl vaccine peptides.” The word “vaccine” in this term does not confer any further limitation on the type of peptides that can be used in methods and compositions of the present invention; rather it is included solely for readability. As described above, bcr-abl vaccine peptides correspond to bcr-abl breakpoint fragments, in some cases containing mutations thereto.

In one embodiment, as described above, bcr-abl peptides of methods and compositions of the present invention correspond to bcr-abl breakpoint fragments. In another embodiment, the bcr-abl breakpoint fragments corresponding to two bcr-abl vaccine peptides (e.g. in the first vaccine mentioned herein, IVHSATGFKQSSKALQRPVASDFE and KALQRPVAS) are distinct from one another.

In another embodiment, the different bcr-abl vaccine peptides correspond to the same bcr-abl breakpoint fragment. For example, in one embodiment of such a vaccine, the unmutated bcr-abl vaccine peptide has the sequence KALQRPVAS (SEQ ID No: 6), and the mutant bcr-abl vaccine peptide has the sequence KLLQRPVAV (SEQ ID No: 7). In both cases, the corresponding bcr-abl breakpoint fragment is KALQRPVAS.

In another embodiment, relevant to vaccines containing 3 or more bcr-abl vaccine peptides, 2 of the bcr-abl vaccine peptides correspond to the same bcr-abl breakpoint fragment, while another bcr-abl vaccine peptide corresponds to a different bcr-abl breakpoint fragment. Each of the above possibilities represents a separate embodiment of the present invention.

In another embodiment, the bcr-abl breakpoint fragments overlap with one another. In one embodiment, the overlap between the bcr-abl breakpoint fragments is at least 7 amino acids (AA). In another embodiment, the overlap is at least 8 AA. In another embodiment, the overlap is at least 9 AA. In another embodiment, the overlap is 7 AA. In another embodiment, the overlap is 8 AA. In another embodiment, the overlap is 9 AA. In another embodiment, the overlap is 10 AA. Each possibility represents a separate embodiment of the present invention.

“Peptide,” in one embodiment of methods and compositions of the present invention, refers to a compound of two or more subunit AA connected by peptide bonds. In another embodiment, the peptide comprises an AA analogue. In another embodiment, the peptide comprises a peptidomimetic. The different AA analogues and peptidomimetics that can be included in the peptides of methods and compositions of the present invention are enumerated hereinbelow. The subunits are, in another embodiment, linked by peptide bonds. In another embodiment, the subunit is linked by another type of bond, e.g. ester, ether, etc. Each possibility represents a separate embodiment of the present invention.

In another embodiment, a peptide of the present invention is immunogenic. In one embodiment, the term “immunogenic” refers to an ability to stimulate, elicit or participate in an immune response. In one embodiment, the immune response elicited is a cell-mediated immune response. In another embodiment, the immune response is a combination of cell-mediated and humoral responses.

In another embodiment, the peptide of methods and compositions of the present invention is so designed as to exhibit affinity for a major histocompatibility complex (MHC) molecule. In one embodiment, the affinity is a high affinity, as described herein.

In another embodiment, T cells that bind to the MHC molecule-peptide complex become activated and induced to proliferate and lyse cells expressing a protein comprising the peptide. T cells are typically initially activated by “professional” antigen presenting cells (“APC”; e.g. dendritic cells, monocytes, and macrophages), which present costimulatory molecules that encourage T cell activation as opposed to anergy or apoptosis. In another embodiment, the response is heteroclitic, as described herein, such that the CTL lyses a neoplastic cell expressing a protein which has an AA sequence homologous to a peptide of this invention, or a different peptide than that used to first stimulate the T cell.

In another embodiment, an encounter of a T cell with a peptide of this invention induces its differentiation into an effector and/or memory T cell. Subsequent encounters between the effector or memory T cell and the same peptide, or, in another embodiment, with a related peptide of this invention, leads to a faster and more intense immune response. Such responses are gauged, in one embodiment, by measuring the degree of proliferation of the T cell population exposed to the peptide. In another embodiment, such responses are gauged by any of the methods enumerated hereinbelow.

In another embodiment, the peptides of methods and compositions of the present invention bind an HLA class I molecule with high affinity. In another embodiment, the peptides bind an HLA class II molecule with high affinity In another embodiment, the peptides bind both an HLA class I molecule and an HLA class II molecule with signficant affinity. In other embodiment, the MHC class I molecule is encoded by any of the HLA-A genes In other embodiment, the MHC class I molecule is encoded by any of the HLA-B genes. In other embodiment, the MHC class I molecule is encoded by any of the HLA-C genes. In another embodiment, the MHC class I molecule is an HLA-0201 molecule. In another embodiment, the molecule is HLA A1. In other embodiments, the molecule is HLA A3.2, HLA A11, HLA A24, HLA B7, HLA B8, HLA B27, or HLA A2, A3, A4, A5, or B8. HLA A1, HLA A2.1, or HLA A3.2. In other embodiment, the MHC class II molecule is encoded by any of the HLA genes HLA-DP, -DQ, or -DR. Each possibility represents a separate embodiment of the present invention.

HLA molecules, known in another embodiment as major histocompatibility complex (MHC) molecules, bind peptides and present them to immune cells. Thus, in another embodiment, the immunogenicity of a peptide is partially determined by its affinity for HLA molecules. HLA class I molecules interact with CD8 molecules, which are generally present on cytotoxic T lymphocytes (CTL). HLA class I molecules interact with CD4 molecules, which are generally present on helper T lymphocytes.

In one embodiment, “affinity” refers to the concentration of peptide necessary for inhibiting binding of a standard peptide to the indicated MHC molecule by fifty percent. In one embodiment, “high affinity” refers to an affinity is such that a concentration of about 500 nanomolar (nM) or less of the peptide is required for inhibition of binding of a standard peptide. In another embodiment, a concentration of about 400 nM or less of the peptide is required. In another embodiment, the binding affinity is 300 nM. In another embodiment, the binding affinity is 200 nM. In another embodiment, the binding affinity is 150 nM. In another embodiment, the binding affinity is 100 nM. In another embodiment, the binding affinity is 80 nM. In another embodiment, the binding affinity is 60 nM. In another embodiment, the binding affinity is 40 nM. In another embodiment, the binding affinity is 30 nM. In another embodiment, the binding affinity is 20 nM. In another embodiment, the binding affinity is 15 nM. In another embodiment, the binding affinity is 10 nM In another embodiment, the binding affinity is 8 nM. In another embodiment, the binding affinity is 6 nM. In another embodiment, the binding affinity is 4 nM In another embodiment, the binding affinity is 3 nM. In another embodiment, the binding affinity is 4 nM. In another embodiment, the binding affinity is 1.5 nM. In another embodiment, the binding affinity is 1 nM. In another embodiment, the binding affinity is 0.8 nM. In another embodiment the binding affinity is 0.6 nM. In another embodiment, the binding affinity is 0.5 nM. In another embodiment, the binding affinity is 0.4 nM. In another embodiment, the binding affinity is 0.3 nM In another embodiment, the binding affinity is less than 0.3 nM.

In another embodiment, “high affinity” refers to a binding affinity of 0.5-500 nM. In another embodiment, the binding affinity is 1-300 nM. In another embodiment, the binding affinity is 1.5-200 nM. In another embodiment, the binding affinity is 2-100 nM. In another embodiment, the binding affinity is 3-100 nM. In another embodiment, the binding affinity is 4-100 nM. In another embodiment, the binding affinity is 6-100 nM. In another embodiment, the binding affinity is 10-100 nM. In another embodiment, the binding affinity is 30-100 nM. In another embodiment, the binding affinity is 3-80 nM. In another embodiment, the binding affinity is 4-60 nM. In another embodiment, the binding affinity is 5-50 nM. In another embodiment, the binding affinity is 6-50 nM. In another embodiment, the binding affinity is 8-50 nM. In another embodiment, the binding affinity is 10-50 nM. In another embodiment, the binding affinity is 20-50 nM. In another embodiment, the binding affinity is 6-40 nM. In another embodiment, the binding affinity is 8-30 nM. In another embodiment, the binding affinity is 10-25 nM. In another embodiment, the binding affinity is 15-25 nM. Each affinity and range of affinities represents a separate embodiment of the present invention.

In another embodiment, the peptides of methods and compositions of the present invention bind to a superfamily of HLA molecules. Superfamilies of HLA molecules share very similar or identical binding motifs. (del Guercio M F, Sidney J, et al, 1995, J Immunol 154: 685-93; Fikes J D, and Sette A, Expert Opin Biol Ther. 2003 September;3(6):985-93). In one embodiment, the superfamily is the A2 superfamily. In another embodiment, the superfamily is the A3 superfamily. In another embodiment, the superfamily is the A24 superfamily. In another embodiment, the superfamily is the B7 superfamily. In another embodiment, the superfamily is the B27 superfamily. In another embodiment, the superfamily is the B44 superfamily. In another embodiment, the superfamily is the C1 superfamily. In another embodiment, the superfamily is the C4 superfamily. In another embodiment, the superfamily is any other superfamily known in the art. Each possibility represents a separate embodiment of the present invention. In one embodiment, the HLA molecule is HLA A0201.

“HLA-binding peptide” refers, in one embodiment, to a peptide that binds an HLA molecule with measurable affinity. In another embodiment, the term refers to a peptide that binds an HLA molecule with high affinity. In another embodiment, the term refers to a peptide that binds an HLA molecule with sufficient affinity to activate a T cell precursor. In another embodiment, the term refers to a peptide that binds an HLA molecule with sufficient affinity to mediate recognition by a T cell. The HLA molecule is, in other embodiments, any of the HLA molecules enumerated herein. Each possibility represents a separate embodiment of the present invention.

As provided herein, bcr-abl breakpoint-derived peptides that stimulated HLA class II molecules, as evidenced by their stimulation of CD4⁺ T cells, were identified (Example 1). In additional experiments, bcr-abl breakpoint-derived peptides with high affinity and low disassociation rate from HLA-A0201 were identified (Examples 2-6). Immunogenicity of some of the peptides was improved by modifying HLA A0201 binding positions. The peptides were found to stimulate T lymphocytes, which produced interferon-γ and induced target cell lysis. The methods disclosed herein will be understood by those in the art to enable design of other bcr-abl breakpoint-derived peptides. The methods further enable design of peptides binding to other HLA molecules. Each possibility represents a separate embodiment of the present invention.

In another embodiment, a bcr-abl vaccine peptide of the present invention is a heteroclitic peptide derived from an bcr-abl breakpoint fragment. In one embodiment, the process of deriving comprises introducing a mutation that enhances a binding of the peptide to an HLA molecule. In another embodiment, the process of deriving consists of introducing a mutation that enhances a binding of the peptide to an MHC class I molecule. Each possibility represents a separate embodiment of the present invention.

“Heteroclitic” refers, in one embodiment, to a peptide that generates an immune response that recognizes the original peptide from which the heteroclitic peptide was derived (e.g. the peptide not containing the anchor residue mutations). In one embodiment, “original peptide” refers to a peptide of the present invention. For example, KLLQRPVAV, (SEQ ID No: 7), was generated from KALQRPVAS (SEQ ID No: 6) by mutation of residues 2 and 9 to leucine and valine, respectively (Examples). In another embodiment, “heteroclitic” refers to a peptide that generates an immune response that recognizes the original peptide from which the heteroclitic peptide was derived, wherein the immune response generated by vaccination with the heteroclitic peptide is greater than the immune response generated by vaccination with the original peptide. In another embodiment, a “heteroclitic” immune response refers to an immune response that recognizes the original peptide from which the improved peptide was derived (e.g. the peptide not containing the anchor residue mutations). In another embodiment, a “heteroclitic” immune response refers to an immune response that recognizes the original peptide from which the heteroclitic peptide was derived, wherein the immune response generated by vaccination with the heteroclitic peptide is greater than the immune response generated by vaccination with the original peptide. Each possibility represents a separate embodiment of the present invention.

In one embodiment, a heteroclitic peptide of the present invention induces an immune response that is increased at least 2-fold relative to the bcr-abl breakpoint peptide from which the heteroclitic peptide was derived. In another embodiment, the increase is 3-fold, or in another embodiment, 5-fold, or in another embodiment, 7-fold, or in another embodiment, 10-fold, or in another embodiment, 20-fold, or in another embodiment, 30-fold, or in another embodiment, 50-fold, or in another embodiment, 100-fold, or in another embodiment, 200-fold, or in another embodiment, 500-fold, or in another embodiment, 1000-fold, or in another embodiment, more than 1000-fold. Each possibility represents a separate embodiment of the present invention.

In another embodiment, a heteroclitic peptide is generated by introduction of a mutation that creates an anchor motif. “Anchor motifs” or “anchor residues” refers, in one embodiment, to one or a set of preferred residues at particular positions in an HLA-binding sequence. In one embodiment, the HLA-binding sequence is an HLA class I-binding sequence. In another embodiment, the positions corresponding to the anchor motifs are those that play a significant role in binding the HLA molecule. In one embodiment, the anchor residue is a primary anchor motif. In another embodiment, the anchor residue is a secondary anchor motif. Each possibility represents a separate embodiment of the present invention.

In another embodiment, the mutation that enhances MHC binding is in the residue at position 1 of the heteroclitic peptide. In one embodiment, the residue is changed to tyrosine. In another embodiment, the residue is changed to glycine. In another embodiment, the residue is changed to threonine. In another embodiment, the residue is changed to phenylalanine. In another embodiment, the residue is changed to any other residue known in the art. In another embodiment, a substitution in position 1 (e.g. to tyrosine) stabilizes the binding of the position 2 anchor residue.

In another embodiment, the mutation is in position 2 of the heteroclitic peptide. In one embodiment, the residue is changed to leucine. In another embodiment, the residue is changed to valine. In another embodiment, the residue is changed to isoleucine. In another embodiment, the residue is changed to methionine. In another embodiment, the residue is changed to any other residue known in the art.

In another embodiment, the mutation is in position 6 of the heteroclitic peptide. In one embodiment, the residue is changed to valine. In another embodiment, the residue is changed to cysteine. In another embodiment, the residue is changed to glutamine. In another embodiment, the residue is changed to histidine. In another embodiment, the residue is changed to any other residue known in the art.

In another embodiment, the mutation is in position 9 of the heteroclitic peptide. In another embodiment, the mutation changes the residue at the C-terminal position thereof. In one embodiment, the residue is changed to valine. In another embodiment, the residue is changed to threonine. In another embodiment, the residue is changed to isoleucine. In another embodiment, the residue is changed to leucine. In another embodiment, the residue is changed to alanine. In another embodiment, the residue is changed to cysteine. In another embodiment, the residue is changed to any other residue known in the art.

In other embodiments, the mutation is in the 3 position, the 4 position, the 5 position, the 7 position, or the 8 position.

Each of the above anchor residues and substitutions represents a separate embodiment of the present invention.

In another embodiment, a bcr-abl vaccine peptide has a length of 8-30 amino acids. In another embodiment, the peptide has a length of 9-11 AA. In another embodiment, the peptide ranges in size from 7-25 AA, or in another embodiment, 8-11, or in another embodiment, 8-15, or in another embodiment, 9-20, or in another embodiment, 9-18, or in another embodiment, 9-15, or in another embodiment, 8-12, or in another embodiment, 9-11 AA in length. In one embodiment the peptide is 8 AA in length, or in another embodiment, 9 AA or in another embodiment, 10 AA or in another embodiment, 12 AA or in another embodiment, 25 AA in length, or in another embodiment, any length therebetween. In another embodiment, the peptide is of greater length, for example 50, or 100, or more. In this embodiment, the cell processes the peptide to a length of 7 and 25 AA in length. In this embodiment, the cell processes the peptide to a length of 9-11 AA Each possibility represents a separate embodiment of the present invention

In another embodiment, the peptide is 15-23 AA in length. In another embodiment, the length is 15-24 AA. In another embodiment, the length is 15-25 AA. In another embodiment, the length is 15-26 AA. In another embodiment, the length is 15-27 AA. In another embodiment, the length is 15-28 AA. In another embodiment, the length is 14-30 AA. In another embodiment, the length is 14-29 AA. In another embodiment, the length is 14-28 AA. In another embodiment, the length is 14-26 AA. In another embodiment, the length is 14-24 AA. In another embodiment, the length is 14-22 AA. In another embodiment, the length is 14-20 AA. In another embodiment, the length is 16-30 AA. In another embodiment, the length is 16-28 AA. In another embodiment, the length is 16-26 AA. In another embodiment, the length is 16-24 AA. In another embodiment, the length is 16-22 AA. In another embodiment, the length is 18-30 AA. In another embodiment, the length is 18-28 AA. In another embodiment, the length is 18-26 AA. In another embodiment, the length is 18-24 AA. In another embodiment, the length is 18-22 AA. In another embodiment, the length is 18-20 AA. In another embodiment, the length is 20-30 AA. In another embodiment, the length is 20-28 AA. In another embodiment, the length is 20-26 AA. In another embodiment, the length is 20-24 AA. In another embodiment, the length is 22-30 AA. In another embodiment, the length is 22-28 AA. In another embodiment, the length is 22-26 AA. In another embodiment, the length is 24-30 AA. In another embodiment, the length is 24-28 AA. In another embodiment, the length is 24-26 AA.

Each of the above peptides, peptide lengths, and types of peptides represents a separate embodiment of the present invention.

As mentioned above, an unmutated bcr-abl vaccine peptide of methods and compositions of the present invention comprises, in one embodiment, an HLA class II-binding peptide. In another embodiment, the unmutated peptide comprises an HLA class I-binding peptide. In another embodiment, the unmutated peptide comprises a peptide that binds another type of HLA molecule. Each possibility represents a separate embodiment of the present invention.

In another embodiment, the HLA class II-binding peptide is an HLA-DRB binding peptide. In another embodiment, the HLA class II-binding peptide is an HLA-DRA binding peptide. In another embodiment, the HLA class II-binding peptide is an HLA-DQA1 binding peptide. In another embodiment, the HLA class II-binding peptide is an HLA-DQB1 binding peptide. In another embodiment, the HLA class II-binding peptide is an HLA-DPA1 binding peptide. In another embodiment, the HLA class II-binding peptide is an HLA-DPB1 binding peptide. In another embodiment, the HLA class II-binding peptide is an HLA-DMA binding peptide. In another embodiment, the HLA class II-binding peptide is an HLA-DMB binding peptide. In another embodiment, the HLA class II-binding peptide is an HLA-DOA binding peptide. In another embodiment, the HLA class II-binding peptide is an HLA-DOB binding peptide. In another embodiment, the HLA class II-binding peptide binds to any other HLA class II molecule known in the art. Each possibility represents a separate embodiment of the present invention.

As mentioned above, a mutant bcr-abl vaccine peptide of methods and compositions of the present invention comprises, in one embodiment, an HLA class I-binding peptide. The HLA class I-binding peptide is, in one embodiment, a degradation product of the mutant bcr-abl vaccine peptide that contains it. For example, in one embodiment of a degradation product, KLLQRPVAV (SEQ ID No: 7) is generated by degradation of SKLLQRPVAVD (SEQ ID No: 25). In another embodiment, the mutant bcr-abl vaccine peptide consists of the HLA class I-binding peptide. Each possibility represents a separate embodiment of the present invention.

“Degradation product” refers, in one embodiment, to a peptide that is generated when a larger peptide is taken up by a cell and digested by intracellular proteases. In another embodiment, “degradation product” refers to a peptide that is generated when a larger peptide is administered to a subject and subsequently digested in vivo. In one embodiment, the digestion is carried out by an intracellular protease In another embodiment, the digestion is carried out by an extracellular protease. In another embodiment, the digestion is carried out by a protease in the plasma, interstitial fluid, or lymph. Each possibility represents a separate embodiment of the present invention

In another embodiment of methods and compositions of the present invention, administration of the mutant bcr-abl vaccine peptide induces an immune response against a cell presenting the bcr-abl breakpoint fragment contained within it. In another embodiment of methods and compositions of the present invention, administration of the mutant bcr-abl vaccine peptide induces an immune response against a cell presenting the HLA class I-binding peptide contained within it. Each possibility represents a separate embodiment of the present invention.

In another embodiment the target cell of the above immune response presents the bcr-abl breakpoint fragment on an HLA molecule. In another embodiment, the HLA molecule is an HLA class I molecule. In other embodiments, the HLA molecule is any HLA class I subtype or HLA class I molecule known in the art. In another embodiment, the immune response against the bcr-abl breakpoint fragment is a heteroclitic immune response Each possibility represents a separate embodiment of the present invention.

In another embodiment, the HLA class I-binding peptide of methods and compositions of the present invention is an HLA-A2 binding peptide. In another embodiment, the HLA class I-binding peptide is an HLA-A3 binding peptide. In another embodiment, the HLA class I-binding peptide is an HLA-A11 binding peptide. In another embodiment, the HLA class I-binding peptide is an HLA-B8 binding peptide. In another embodiment, the HLA class I-binding peptide is an HLA-0201 binding peptide. In another embodiment, the HLA class I-binding peptide binds any other HLA class I molecule known in the art. Each possibility represents a separate embodiment of the present invention.

In another embodiment, a vaccine of methods and compositions of the present invention further comprises an additional unmutated bcr-abl vaccine peptide. In another embodiment, the additional unmutated bcr-abl vaccine peptide corresponds to an additional bcr-abl breakpoint fragment.

For example, in one embodiment of such a vaccine, the additional unmutated bcr-abl vaccine peptide has the sequence KQSSKALQR (SEQ ID No: 3), in addition to IVHSATGFKQSSKALQRPVASDFE (the first unmutated bcr-abl vaccine peptide; SEQ ID No: 18) and KLLQRPVAV (the mutant bcr-abl vaccine peptide; SEQ ID No: 7). KQSSICALQR is also, in this embodiment, the sequence of the bcr-abl breakpoint fragment that corresponds to the additional unmutated bcr-abl vaccine peptide. Thus, 3 bcr-abl breakpoint fragments correspond to the bcr-abl vaccine peptides of this vaccine; namely, KQSSKALQR and the first and second bcr-abl breakpoint fragments, corresponding to the other bcr-abl vaccine peptides, IVHSATGFKQSSKALQRPVASDFE and KLLQRPVAV, respectively. Each possibility represents a separate embodiment of the present invention.

In another embodiment, a vaccine of methods and compositions of the present invention further comprises an additional mutant bcr-abl vaccine peptide. In another embodiment, the additional mutant bcr-abl vaccine peptide comprises an additional HLA class I-binding peptide, wherein the additional HLA class I-binding peptide corresponds to an additional bcr-abl breakpoint fragment, with a mutation in an anchor residue of the additional bcr-abl breakpoint fragment.

For example, in one embodiment of such a vaccine, the additional mutant bcr-abl vaccine peptide has the sequence YLKALQRPV (SEQ ID No: 2), in addition to IVHSATGFKQSSKALQRPVASDFE (the first unmutated bcr-abl vaccine peptide; SEQ ID No: 18); KLLQRPVAV (the first mutant bcr-abl vaccine peptide; SEQ ID No: 7); and KQSSKALQR (the second unmutated bcr-abl vaccine peptide; SEQ ID No: 3). In this embodiment, SSKALQRPV (SEQ ID No: 1) is the sequence of the bcr-abl breakpoint fragment that corresponds to the additional mutant bcr-abl vaccine peptide. YLKALQRPV is derived from SSKALQRPV by mutation of residues 1 and 2 to tyrosine and leucine, respectively. Thus, 4 bcr-abl breakpoint fragments correspond to the bcr-abl vaccine peptides of this vaccine; namely, SSKALQRPV and the first, second, and third bcr-abl breakpoint fragments, corresponding to the other bcr-abl vaccine peptides in the vaccine.

In another embodiment of the above vaccine, a third mutant bcr-abl vaccine peptide is included For example, in one embodiment of such a vaccine, the third mutant bcr-abl vaccine peptide has the sequence GFKQSSKAL (SEQ ID No: 19), in addition to IVHSATGFKQSSKALQRPVASDFE, KLLQRPVAV, KQSSKALQR, and YLKALQRPV. In this embodiment, the third mutant bcr-abl vaccine peptide corresponds to a fifth bcr-abl breakpoint fragment, in addition to the first, second, third, and fourth bcr-abl breakpoint fragments, corresponding to the other bcr-abl vaccine peptides in the vaccine.

In another embodiment, a vaccine of methods and compositions of the present invention contains one unmutated bcr-abl vaccine peptide and more than one mutant bcr-abl vaccine peptide. For example, in one embodiment of such a vaccine, the additional mutant bcr-abl vaccine peptide has the sequence YLKALQRPV (SEQ ID No: 2), in addition to IVHSATGFKQSSKALQRPVASDFE (the unmutated bcr-abl vaccine peptide; SEQ ID No: 18); and KLLQRPVAV (the first mutant bcr-abl vaccine peptide; SEQ ID No: 7). In this embodiment, SSKALQRPV (SEQ ID No: 1) is the sequence of the bcr-abl breakpoint fragment corresponding to the additional mutant bcr-abl vaccine peptide Thus, 3 bcr-abl breakpoint fragments correspond to the bcr-abl vaccine peptides of this vaccine; namely, SSKALQRPV and the first and second bcr-abl breakpoint fragments, corresponding to the other bcr-abl vaccine peptides in the vaccine.

Each of the above combinations of peptides represents a separate embodiment of the present invention.

All the embodiments enumerated above for the exemplary vaccine mentioned above are applicable, in other embodiments, to each of the vaccines mentioned below, and to each method comprising same. Each possibility represents a separate embodiment of the present invention.

In another embodiment, a bcr-abl vaccine of methods and compositions of the present invention is a b3a2 vaccine. In one embodiment of this vaccine, the bcr-abl breakpoint fragments corresponding to the peptides of the vaccine are b3a2 breakpoint fragments. Each possibility represents a separate embodiment of the present invention.

In another embodiment, an unmutated b3a2 vaccine peptide of methods and compositions of the present invention has an AA sequence comprising IVHSATGFKQSSKALQRPVASDFE (SEQ ID No: 18) In another embodiment, the AA sequence is IVHSATGFKQSSKALQRPVASDFE. In another embodiment, the AA sequence is IVHSATGFKQSSKALQRPVASDFEP (SEQ ID No: 24). In another embodiment, the AA sequence comprises KQSSKALQR (SEQ ID No: 3). In another embodiment, the AA sequence is KQSSKALQR. In another embodiment, the AA sequence comprises GFKQSSKAL (SEQ ID No: 19). In another embodiment, the AA sequence is GFKQSSKAL. In another embodiment, the AA sequence is a fragment of IVHSATGFKQSSKALQRPVASDFE. In another embodiment, the unmutated b3a2 peptide has any other b3a2 breakpoint sequence known in the art. Each possibility represents a separate embodiment of the present invention.

A mutated b3a2 vaccine peptide of methods and compositions of the present invention has, in one embodiment, an AA sequence comprising KLLQRPVAV (SEQ ID No: 7). In another embodiment, the AA sequence is KLLQRPVAV (SEQ ID No: 7). In another embodiment, the AA sequence comprises YLKALQRPV (SEQ ID No: 2). In another embodiment, the AA sequence is YLKALQRPV (SEQ ID No: 2). In another embodiment, the AA sequence is TLFKQSSKV (SEQ ID No: 9) In another embodiment, the AA sequence comprises TLFKQSSKV. In another embodiment, the AA sequence is YLFKQSSKV (SEQ ID No: 10). In, another embodiment, the AA sequence comprises YLFKQSSKV. Each possibility represents a separate embodiment of the present invention.

In another embodiment, a bcr-abl breakpoint fragment corresponding to a mutated b3a2 peptide of methods and compositions of the present invention has the AA sequence SSKALQRPV (SEQ ID No: 1). In another embodiment, the bcr-abl breakpoint fragment has the AA sequence KQSSKALQR (SEQ ID No: 3) In another embodiment, the AA sequence is KALQRPVAS (SEQ ID No: 6). In another embodiment, the AA sequence is TGFKQSSKA (SEQ ID No: 8). In another embodiment, the AA sequence is SKALQRPV (SEQ ID No: 26). In another embodiment, the AA sequence is KQSSKALQRPV (SEQ ID No: 27). In another embodiment, the AA sequence is QSSKALQRPV, (SEQ ID No: 28). Each possibility represents a separate embodiment of the present invention.

In another embodiment, a b3a2 vaccine of methods and compositions of the present invention further comprises an additional unmutated bcr-abl vaccine peptide. In one embodiment, the additional unmutated bcr-abl vaccine peptide has an AA sequence comprising IVHSATGFKQSSKALQRPVASDFE (SEQ ID No: 18). In another embodiment, the AA sequence is IVHSATGFKQSSKALQRPVASDFE. In another embodiment, the AA sequence comprises KQSSKALQR (SEQ ID No: 3). In another embodiment, the AA sequence is KQSSKALQR. In another embodiment, the AA sequence comprises GFKQSSKAL (SEQ ID No: 19). In another embodiment, the AA sequence is GFKQSSKAL. In another embodiment, the AA sequence comprises ATGFKQSSKALQRPVAS (SEQ ID No: 23). In another embodiment, the AA sequence is ATGFKQSSKALQRPVAS. Each possibility represents a separate embodiment of the present invention.

In another embodiment, a b3a2 vaccine of methods and compositions of the present invention further comprises an additional mutant bcr-abl vaccine peptide. In one embodiment, the additional mutant bcr-abl vaccine peptide comprises an additional HLA class I-binding peptide, in addition to the HLA class I-binding peptide contained in the first mutant bcr-abl vaccine peptide. In another embodiment, the additional mutant bcr-abl vaccine peptide has an AA sequence comprising KLLQRPVAV (SEQ ID No: 7). In another embodiment, the AA sequence is KLLQRPVAV. In another embodiment, the AA sequence comprises YLKALQRPV (SEQ ID No: 2). In another embodiment, the AA sequence is YLKALQRPV. In another embodiment, the bcr-abl breakpoint fragment corresponding to the additional mutant bcr-abl vaccine peptide has the AA sequence SSKALQRPV (SEQ ID No: 1). In another embodiment, the bcr-abl breakpoint fragment has the AA sequence KALQRPVAS (SEQ ID No: 6). Each possibility represents a separate embodiment of the present invention.

Each of the above unmutated b3a2 vaccine peptides and mutant b3a2 vaccine peptides, and each combination thereof, represents a separate embodiment of the present invention.

In another embodiment, a bcr-abl vaccine of methods and compositions of the present invention is a b2a2 vaccine. In one embodiment of this vaccine, the bcr-abl breakpoint fragments corresponding to the peptides of the vaccine are b2a2 breakpoint fragments.

In another embodiment, an unmutated b2a2 vaccine peptide of methods and compositions of the present invention has an AA sequence comprising VHSIPLTINKEEALQRPVASDFE (SEQ ID No: 17). In another embodiment, the AA sequence is VHSIPLTINKEEALQRPVASDFE. In another embodiment, the AA sequence comprises the sequence IPLTINKEEALQRPVAS (SEQ ID No: 20). In another embodiment, the AA sequence is IPLTINKEEALQRPVAS.

In another embodiment, a mutant b2a2 vaccine peptide of methods and compositions of the present invention has an AA sequence comprising YLINKEEAL (SEQ ID No: 14). In another embodiment, the AA sequence is YLINKEEAL. In another embodiment, the AA sequence is YLINKEEAV (SEQ ID No: 15). In another embodiment, the AA sequence comprises YLINKEEAV. In another embodiment, the AA sequence is YLINKVEAL (SEQ ID No: 16). In another embodiment, the AA sequence comprises YLINKVEAL.

In another embodiment, the bcr-abl breakpoint fragment corresponding to the mutant bcr-abl vaccine peptide has the AA sequence LTINKEEAL, (SEQ ID No: 11). In another embodiment, the AA sequence comprises LTINIKEEAL.

Each of the above unmutated b2a2 vaccine peptides, mutant b2a2 vaccine peptides, bcr-abl breakpoint fragments, and each combination thereof, represents a separate embodiment of the present invention.

In another embodiment, a bcr-abl vaccine of methods and compositions of the present invention is a vaccine against a bcr-abl protein created by a translocation other than b3a2 or b2a2 (e.g. p 185-190^{bcr-abl}) The bcr-abl protein is, in other embodiments, a result of any translocation known in the art that generates a bcr-abl protein. Each possibility represents a separate embodiment of the present invention.

In another embodiment, the present invention provides a bcr-abl vaccine comprising peptides having the sequences VHSIPLTINKEEALQRPVASDFE (SEQ ID No: 17) and YLINKEEAL (SEQ ID No: 14). In another embodiment, the bcr-abl vaccine further comprises an adjuvant.

In another embodiment, the present invention provides a bcr-abl vaccine comprising peptides having the sequences IVHSATGFKQSSKALQRPVASDFE (SEQ ID No: 18), KQSSKALQR (SEQ ID No: 3), GFKQSSKAL (SEQ ID No: 19), KLLQRPVAV (SEQ ID No: 7), and YLKALQRPV (SEQ ID No: 2) In another embodiment, the bcr-abl vaccine further comprises an adjuvant.

In another embodiment, minor modifications are made to peptides of the present invention without decreasing their affinity for HLA molecules or changing their TCR specificity, utilizing principles well known in the art. “Minor modifications,” in one embodiment, refers to e.g. insertion, deletion, or substitution of one AA, inclusive, or deletion or addition of 1-3 AA outside of the residues between 2 and 9, inclusive. While the computer algorithms described herein are useful for predicting the MHC class I-binding potential of peptides, they have 60-80% predictive accuracy; and thus, the peptides should be evaluated empirically before a final determination of MHC class I-binding affinity is made. Thus, peptides of the present invention are not limited to peptides predicated by the algorithms to exhibit strong MHC class I-binding affinity. The types are modifications that can be made are listed below. Each modification represents a separate embodiment of the present invention.

In another embodiment, a peptide enumerated in the Examples of the present invention is further modified by mutating an anchor residue to an MHC class I preferred anchor residue, which can be, in other embodiments, any of the anchor residues enumerated herein. In another embodiment, a peptide of the present invention containing an MHC class I preferred anchor residue is further modified by mutating the anchor residue to a different MHC class I preferred residue for that location. The different preferred residue can be, in other embodiments, any of the preferred residues enumerated herein.

In one embodiment, the anchor residue that is further modified is in the 1 position. In another embodiment, the anchor residue is in the 2 position. In another embodiment, the anchor residue is in the 3 position. In another embodiment, the anchor residue is in the 4 position. In another embodiment, the anchor residue is in the 5 position. In another embodiment, the anchor residue is in the 6 position. In another embodiment, the anchor residue is in the 7 position. In another embodiment, the anchor residue is in the 8 position. In another embodiment, the anchor residue is in the 9 position. Residues other than 2 and 9 can also serve as secondary anchor residues; therefore, mutating them can improve MHC class I binding. Each possibility represents a separate embodiment of the present invention.

In another embodiment, a peptide of methods and compositions of the present invention is a length variant of a peptide enumerated in the Examples. In one embodiment, the length variant is one amino acid (AA) shorter than the peptide from the Examples. In another embodiment, the length variant is two AA shorter than the peptide from the Examples. In another embodiment, the length variant is more than two AA shorter than the peptide from the Examples. In another embodiment, the shorter peptide is truncated on the N-terminal end. In another embodiment, the shorter peptide is truncated on the C-terminal end. In another embodiment, the truncated peptide is truncated on both the N-terminal and C-terminal ends. Peptides are, in one embodiment, amenable to truncation without changing affinity for HLA molecules, as is well known in the art. In other embodiments, the truncated peptide has one of the sequences:

| | |
|---|---|
| HSIPLTINKEEALQRPVASDFE, | (SEQ ID No: 31-50) |
| HSIPLTINKEEALQRPVASDF, | |
| VHISIPLTINKEEALQRPVASDF, | |
| SIPLTINKEEALQRPVASDFE, | |
| VHSIPLTINKEEALQRPVASD, | |
| LINKEEAL, | |
| YLINKEEA, | |
| VHSATGFKQSSKALQRPVASDFE, | |
| VHSATGFKQSSKALQRPVASDF | |
| IVHSATGFKQSSKALQRPVASDF, | |
| HSATGFKQSSKALQRPVASDFE, | |
| IVHSATGFKQSSKALQRPVASD, | |
| QSSKALQR, | |
| KQSSKALQ, | |
| FKQSSKAL, | |
| GFKQSSKA, | |
| LLQRPVAV, | |
| KLLQRPVA, | |
| LKALQRPV, | |
| or | |
| YLKALQRP. | |

Each of the above truncated peptides represents a separate embodiment of the present invention.

In another embodiment, the length variant is longer than a peptide enumerated in the Examples of the present invention In another embodiment, the longer peptide is extended on the N-terminal end in accordance with the surrounding bcr-abl sequence. Peptides are, in one embodiment, amenable to extension on the N-terminal end without changing affinity for HLA molecules, as is well known in the art. Such peptides are thus equivalents of the peptides enumerated in the Examples. In another embodiment, the N-terminal extended peptide is extended by one residue. In another embodiment, the N-terminal extended peptide is extended by two residues. In another embodiment, the N-terminal extended peptide is extended by three residues. In another embodiment, the N-terminal extended peptide is extended by more than three residues.

In other embodiments, the N-terminal extended peptide has one of the sequences:

| | |
|---|---|
| KLQTVHSIPLTINKEEALQRPVASDFE, | (SEQ ID No: 51-63) |
| LQTVHSIPLTINKEEALQRPVASDFE, | |
| QTVHSIPLTINKEEALQRPVASDFE, | |
| TVHSIPLTINKEEALQRPVASDFE, | |
| PYLINKEEAL, | |
| FLNVIVHSATGFKQSSKALQRPVASDFE, | |
| LNVIVHSATGFKQSSKALQRPVASDFE, | |
| NVIVHSATGFKQSSKALQRPVASDFE, | |
| VIVHSATGFKQSSKALQRPVASDFE, | |
| FKQSSKALQR, | |
| TGFKQSSKAL, | |
| SKLLQRPVAV, | |
| or | |
| QYLKALQRPV, | |

In one embodiment, the longer peptide is extended on the C terminal end in accordance with the surrounding bcr-abl sequence. Peptides are, in one embodiment, amenable to extension on the C-terminal end without changing affinity for HLA molecules, as is well known in the art. Such peptides are thus equivalents of the peptides enumerated in the Examples of the present invention. In another embodiment, the C-terminal extended peptide is extended by one residue. In another embodiment, the C-terminal extended peptide is extended by two residues. In another embodiment, the C-terminal extended peptide is extended by three residues. In another embodiment, the C-terminal extended peptide is extended by more than three residues. In other embodiments, the peptide has one of the sequences:

| | |
|---|---|
| VHSIPLTINKEEALQRPVASDFEPQGL, | (SEQ ID No: 64-81) |
| VHSIPLTINKEEALQRPVASDFEPQG, | |
| VHSIPLTINKEEALQRPVASDFEPQ, | |
| VHSIPLTINKEEALQRPVASDFEP, | |
| YLINKEEALQR, | |
| YLINKEEALQ, | |
| IVHSATGFKQSSKALQRPVASDFEPQGL, | |
| IVHSATGFKQSSKALQRPVASDFEPQG, | |
| IVHSATGFKQSSKALQRPVASDFEPQ, | |
| KQSSKALQRPV, | |
| KQSSKALQRP, | |
| GFKQSSKALQR, | |
| GFKQSSKALQ, | |
| KLLQRPVAVDF, | |
| KLLQRPVAVD, | |
| YLKALQRPVAS, | |
| or | |
| YLKALQRPVA. | |

In another embodiment, the extended peptide is extended on both the N-terminal and C-terminal ends. In another embodiment, the extended peptide has one of the following sequences:

(SEQ ID No: 82-96)

KLQTVHSIPLTINKEEALQRPVASDFEPQGL,
KLQTVHSIPLTINKEEALQRPVASDFEP,
KLQTVHSIPLTINKEEALQRPVASDFEPQ,
KLQTVHSIPLTINKEEALQRPVASDFEPQG,
TVHSIPLTINKEEALQRPVASDFEPQGL,
QTVHSIPLTINKEEALQRPVASDFEPQGL,
LQTVHSIPLTINKEEALQRPVASDFEPQGL,
FLNVIVHSATGFKQSSKALQRPVASDFEPQGL,
FLNVIVHSATGFKQSSKALQRPVASDFEP,
FLNVIVHSATGFKQSSKALQRPVASDFEPQ,
FLNVIVHSATGFKQSSKALQRPVASDFEPQG,
VIVHSATGFKQSSKALQRPVASDFEPQGL,
NVIVHSATGFKQSSKALQRPVASDFEPQGL,
or
LNVIVHSATGFKQSSKALQRPVASDFEPQGL.

Each of the above extended peptides represents a separate embodiment of the present invention.

In another embodiment, a truncated peptide of the present invention retains the HLA anchor residues on the second residue and the C-terminal residue, with a smaller number of intervening residues (e.g. 5) than a peptide enumerated in the Examples of the present invention. Peptides are, in one embodiment, amenable to such mutation without changing affinity for HLA molecules. In one embodiment, such a truncated peptide is designed by removing one of the intervening residues of one of the above sequences. In another embodiment, the HLA anchor residues are retained on the second and eighth residues. In another embodiment, the HLA anchor residues are retained on the first and eighth residues. Each possibility represents a separate embodiment of the present invention.

In another embodiment, an extended peptide of the present invention retains the HLA anchor residues on the second residue and the C-terminal residue, with a larger number of intervening residues (e.g. 7 or 8) than a peptide enumerated in the Examples of the present invention. In one embodiment, such an extended peptide is designed by adding one or more residues between two of the intervening residues of one of the above sequences. It is well known in the art that residues can be removed from or added between the intervening sequences of HLA-binding peptides without changing affinity for HLA. Such peptides are thus equivalents of the peptides enumerated in the Examples of the present invention. In another embodiment, the HLA anchor residues are retained on the second and ninth residues. In another embodiment, the HLA anchor residues are retained on the first and eighth residues. In another embodiment, the HLA anchor residues are retained on the two residues separated by six intervening residues. Each possibility represents a separate embodiment of the present invention.

In another embodiment, a peptide of the present invention is homologous to a peptide enumerated in the Examples. The terms “homology,” “homologous,” etc, when in reference to any protein or peptide, refer, in one embodiment, to a percentage of amino acid residues in the candidate sequence that are identical with the residues of a corresponding native polypeptide, after aligning the sequences and introducing gaps, if necessary, to achieve the maximum percent homology, and not considering any conservative substitutions as part of the sequence identity. Methods and computer programs for the alignment are well known in the art.

In another embodiment, the term “homology,” when in reference to any nucleic acid sequence similarly indicates a percentage of nucleotides in a candidate sequence that are identical with the nucleotides of a corresponding native nucleic acid sequence.

Homology is, in one embodiment, determined by computer algorithm for sequence alignment, by methods well described in the art. In other embodiments, computer algorithm analysis of nucleic acid sequence homology includes the utilization of any number of software packages available, such as, for example, the BLAST, DOMAIN, BEAUTY (BLAST Enhanced Alignment Utility), GENPEPT and TREMBL packages.

In another embodiment, “homology” refers to identity to a sequence selected from SEQ ID No: 1-96 of greater than 70%. In another embodiment, “homology” refers to identity to a sequence selected from SEQ ID No: 1-96 of greater than 72%. In another embodiment, “homology” refers to identity to one of SEQ ID No: 1-96 of greater than 75%. In another embodiment, “homology” refers to identity to a sequence selected from SEQ ID No: 1-96 of greater than 78%. In another embodiment, “homology” refers to identity to one of SEQ ID No: 1-96 of greater than 80%. In another embodiment, “homology” refers to identity to one of SEQ ID No: 1-96 of greater than 82%. In another embodiment, “homology” refers to identity to a sequence selected from SEQ ID No: 1-96 of greater than 83%. In another embodiment, “homology” refers to identity to one of SEQ ID No: 1-96 of greater than 85%. In another embodiment, “homology” refers to identity to one of SEQ ID No: 1-96 of greater than 87%. In another embodiment, “homology” refers to identity to a sequence selected from SEQ ID No: 1-96 of greater than 88%. In another embodiment, “homology” refers to identity to one of SEQ ID No: 1-96 of greater than 90%. In another embodiment, “homology” refers to identity to one of SEQ ID No: 1-96 of greater than 92%. In another embodiment, “homology” refers to identity to a sequence selected from SEQ ID No: 1-96 of greater than 93%. In another embodiment, “homology” refers to identity to one of SEQ ID No: 1-96 of greater than 95%. In another embodiment, “homology” refers to identity to a sequence selected from SEQ ID No: 1-96 of greater than 96%. In another embodiment, “homology” refers to identity to one of SEQ ID No: 1-96 of greater than 97%. In another embodiment, “homology” refers to identity to one of SEQ ID No: 1-96 of greater than 98%. In another embodiment, “homology” refers to identity to one of SEQ ID No: 1-96 of greater than 99%, In another embodiment, “homology” refers to identity to one of SEQ ID No: 1-96 of 100%. Each possibility represents a separate embodiment of the present invention.

In another embodiment, homology is determined is via determination of candidate sequence hybridization, methods of which are well described in the art (See, for example, “Nucleic Acid Hybridization” Hames, B. D., and Higgins S. J., Eds. (1985); Sambrook et al., 2001, Molecular Cloning, A Laboratory Manual, Cold Spring Harbor Press, N.Y.; and Ausubel et al., 1989, Current Protocols in Molecular Biology, Green Publishing Associates and Wiley Interscience, N.Y.). In another embodiments, methods of hybridization are carried out under moderate to stringent conditions, to the complement of a DNA encoding a native caspase peptide. Hybridization conditions being, for example, overnight incubation at 42° C. in a solution comprising: 10-20% formamide, 5×SSC (150 mM NaCl, 15 mM trisodium citrate), 50 mM sodium phosphate (pH 7.6), 5× Denhardt's solution, 10% dextran sulfate, and 20 μg/ml denatured, sheared salmon sperm DNA.

Each of the above homologues and variants of peptides enumerated in the Examples represents a separate embodiment of the present invention.

In another embodiment, the present invention provides a composition comprising a peptide of this invention. In another embodiment, the composition further comprises a pharmaceutically acceptable carrier. In another embodiment, the composition further comprises an adjuvant. In another embodiment, the composition comprises two or more peptides of the present invention. In another embodiment, the composition further comprises any of the additives, compounds, or excipients set forth hereinbelow. In one embodiment, the adjuvant is QS21, Freund's complete or incomplete adjuvant, aluminum phosphate, aluminum hydroxide, BCG or alum. In other embodiments, the carrier is any carrier enumerated herein. In other embodiments, the adjuvant is any adjuvant enumerated herein. Each possibility represents a separate embodiment of the present invention.

In another embodiment, this invention provides a vaccine comprising a peptide of this invention, which in another embodiment further comprises a carrier, adjuvant, or combination thereof.

In another embodiment, the term “vaccine” refers to a material or composition that, when introduced into a subject, provides a prophylactic or therapeutic response for a particular disease, condition, or symptom of same. In another embodiment, this invention comprises peptide-based vaccines, wherein the peptide comprises any embodiment listed herein, including immunomodulating compounds such as cytokines, adjuvants, etc.

It is to be understood that any embodiments described herein, regarding peptides, vaccines and compositions of this invention can be employed in any of the methods of this invention. Each combination of peptide, vaccine, or composition with a method represents an embodiment thereof.

In another embodiment, a bcr-abl vaccine of methods and compositions of the present invention further comprises an adjuvant. In one embodiment, the adjuvant is Montamide ISA 51. Montamide ISA 51 contains a natural metabolizable oil and a refined emulsifier. In another embodiment, the adjuvant is GM-CSF. Recombinant GM-CSF is a human protein grown, in one embodiment, in a yeast (S. cerevisiae) vector. GM-CSF promotes clonal expansion and differentiation of hematopoietic progenitor cells, APC, and dendritic cells and T cells.

In another embodiment, the adjuvant is a cytokine. In another embodiment, the adjuvant is a growth factor. In another embodiment, the adjuvant is a cell population. In another embodiment, the adjuvant is QS21. In another embodiment, the ‘adjuvant is Freund’s incomplete adjuvant. In another embodiment, the adjuvant is aluminum phosphate. In another embodiment, the adjuvant is aluminum hydroxide. In another embodiment, the adjuvant is BCG. In another embodiment, the adjuvant is alum. In another embodiment, the adjuvant is an interleukin. In another embodiment, the adjuvant is a chemokine. In another embodiment, the adjuvant is any other type of adjuvant known in the art. In another embodiment, the bcr-abl vaccine comprises two the above adjuvants. In another embodiment, the bcr-abl vaccine comprises more than two the above adjuvants. Each possibility represents a separate embodiment of the present invention.

In another embodiment, the present invention provides a method of treating a subject with a bcr-abl-associated cancer, the method comprising administering to the subject a bcr-abl vaccine of the present invention, thereby treating a subject with a bcr-abl-associated cancer.

In another embodiment, the present invention provides a method of suppressing or halting the progression of a bcr-abl-associated cancer in a subject, the method comprising administering to the subject a bcr-abl vaccine of the present invention, thereby suppressing or halting the progression of a bcr-abl-associated cancer.

In another embodiment, the present invention provides a method of reducing the incidence of a bcr-abl-associated cancer in a subject, the method comprising administering to the subject a bcr-abl vaccine of the present invention, thereby reducing the incidence of a bcr-abl-associated cancer in a subject.

In another embodiment, the present invention provides a method of reducing the incidence of relapse of a bcr-abl-associated cancer in a subject, the method comprising administering to the subject a bcr-abl vaccine of the present invention, thereby reducing the incidence of relapse of a bcr-abl-associated cancer in a subject.

In another embodiment, the present invention provides a method of breaking a T cell tolerance of a subject to a bcr-abl-associated cancer, the method comprising administering to the subject a bcr-abl vaccine of the present invention, thereby breaking a T cell tolerance to a bcr-abl-associated cancer.

In another embodiment, the present invention provides a method of treating a subject with a cancer associated with a b3a2 bcr-abl chromosomal translocation, the method comprising administering to the subject a b3a2 bcr-abl vaccine of the present invention, thereby treating a subject with a cancer associated with a b3a2 bcr-abl chromosomal translocation.

In another embodiment, the present invention provides a method of reducing the incidence of a cancer in a subject, wherein the cancer is associated with a b3a2 bcr-abl chromosomal translocation, the method comprising administering to the subject a b3a2 bcr-abl vaccine of the present invention, thereby reducing the incidence of a cancer associated with a b3a2 bcr-abl chromosomal translocation in a subject.

In another embodiment, the present invention provides a method of reducing the incidence of relapse of a cancer in a subject, wherein the cancer is associated with a b3a2 bcr-abl chromosomal translocation, the method comprising administering to the subject a b3a2 bcr-abl vaccine of the present invention, thereby reducing the incidence of relapse of a cancer associated with a b3a2 bcr-abl chromosomal translocation in a subject.

In another embodiment, the present invention provides a method of treating a subject with a cancer associated with a b2a2 bcr-abl chromosomal translocation, the method comprising administering to the subject a b2a2 bcr-abl vaccine of the present invention, thereby treating a subject with a cancer associated with a b2a2 bcr-abl chromosomal translocation.

In another embodiment, the present invention provides a method of reducing the incidence of a cancer in a subject, wherein the cancer is associated with a b2a2 bcr-abl chromosomal translocation, the method comprising administering to the subject a b2a2 bcr-abl vaccine of the present invention, thereby reducing the incidence of a cancer associated with a b2a2 bcr-abl chromosomal translocation in a subject.

In another embodiment, the present invention provides a method of reducing the incidence of relapse of a cancer in a subject, wherein the cancer is associated with a b2a2 bcr-abl chromosomal translocation, the method comprising administering to the subject a b2a2 bcr-abl vaccine of the present invention, thereby reducing the incidence of relapse of a cancer associated with a b2a2 bcr-abl chromosomal translocation in a subject.

In another embodiment, the present invention provides a method of treating a subject having a bcr-abl-associated cancer, comprising (a) inducing in a donor formation and proliferation of human cytotoxic T lymphocytes (CTL) that recognize a malignant cell of the cancer by a method of the present invention; and (b) infusing the human CTL into the subject, thereby treating a subject having a cancer.

In another embodiment, the present invention provides a method of treating a subject having a bcr-abl-associated cancer, comprising (a) inducing ex vivo formation and proliferation of human CTL that recognize a malignant cell of the cancer by a method of the present invention, wherein the human immune cells are obtained from a donor; and (b) infusing the human CTL into the subject, thereby treating a subject having a cancer.

In another embodiment, the present invention provides a method of inducing the formation and proliferation of CTL specific for cancer cells that are associated with a bcr-abl translocation, the method comprising contacting a lymphocyte population with a vaccine of the present invention. In one embodiment, the vaccine is an antigen presenting cell (APC) associated with a mixture of peptides of the present invention.

In another embodiment, this invention provides a method of generating a heteroclitic immune response in a subject, wherein the heteroclitic immune response is directed against a cancer associated with a bcr-abl translocation, the method comprising administering to the subject a vaccine of the present invention, thereby generating a heteroclitic immune response.

In another embodiment, this invention provides a method of reducing the number of cancer cells in a subject having CML, the method comprising administering to the subject a vaccine of the present invention, thereby reducing the number of cancer cells in a subject having CML.

Any embodiments enumerated herein, regarding peptides, vaccines and compositions of this invention can be employed in any of the methods of this invention, and each represents an embodiment thereof.

In another embodiment, multiple peptides of this invention are used to stimulate an immune response in methods of the present invention.

In one embodiment, the bcr-abl-associated cancer treated by a method of the present invention is acute myeloid leukemia (AML). In another embodiment, the bcr-abl-associated cancer is chronic myeloid leukemia (CML). In another embodiment, the bcr-abl-associated cancer is acute lymphoblastic leukemia (ALL). In another embodiment, the bcr-abl-associated cancer is any other bcr-abl-associated cancer known in the art.

In another embodiment, a malignant cell of the bcr-abl-associated cancer presents a bcr-abl breakpoint fragment corresponding to a bcr-abl vaccine peptide of the vaccine on an HLA class I molecule thereof. Each possibility represents a separate embodiment of the present invention.

In another embodiment, a mutant bcr-abl vaccine peptide of a vaccine of methods and compositions of the present invention comprises an HLA class II-binding peptide. In another embodiment, the HLA class II-binding peptide corresponds to a bcr-abl breakpoint fragment with a mutation in HLA class II molecule anchor residue.

In one embodiment, methods of the present invention provide for an improvement in an immune response that has already been mounted by a subject. In one embodiment, methods of the present invention comprise administering the peptide, composition, or vaccine 2 or more times. In another embodiment, the peptides are varied in their composition, concentration, or a combination thereof. In another embodiment, the peptides provide for the initiation of an immune response against an antigen of interest in a subject in which an immune response against the antigen of interest has not already been initiated. In another embodiment, the CTL that are induced proliferate in response to presentation of the peptide on the APC or cancer cell. In other embodiments, reference to modulation of the immune response involves, either or both the humoral and cell-mediated arms of the immune system, which is accompanied by the presence of Th2 and Th1 T helper cells, respectively, or in another embodiment, each arm individually.

In other embodiments, the methods affecting the growth of a tumor result in (1) the direct inhibition of tumor cell division, or (2) immune cell mediated tumor cell lysis, or both, which leads to a suppression in the net expansion of tumor cells.

Inhibition of tumor growth by either of these two mechanisms can be readily determined by one of ordinary skill in the art based upon a number of well known methods. In one embodiment, tumor inhibition is determined by measuring the actual tumor size over a period of time. In another embodiment, tumor inhibition can be determined by estimating the size of a tumor (over a period of time) utilizing methods well known to those of skill in the art. More specifically, a variety of radiologic imaging methods (e.g., single photon and positron emission computerized tomography; see generally, “Nuclear Medicine in Clinical Oncology,” Winkler, C. (ed.) Springer-Verlag, New York, 1986), can be utilized to estimate tumor size. Such methods can also utilize a variety of imaging agents, including for example, conventional imaging agents (e.g., Gallium-67 citrate), as well as specialized reagents for metabolite imaging, receptor imaging, or immunologic imaging (e.g., radiolabeled monoclonal antibody specific tumor markers). In addition, non-radioactive methods such as ultrasound (see, “Ultrasonic Differential Diagnosis of Tumors”, Kossoff and Fukuda, (eds.), Igaku-Shoin, New York, 1984), can also be utilized to estimate the size of a tumor.

In addition to the in vivo methods for determining tumor inhibition discussed above, a variety of in vitro methods can be utilized in order to predict in vivo tumor inhibition. Representative examples include lymphocyte mediated anti-tumor cytolytic activity determined for example, by a ⁵¹Cr release assay (Examples), tumor dependent lymphocyte proliferation (Ioannides, et al., J. Immunol. 146(5):1700-1707, 1991), in vitro generation of tumor specific antibodies (Herlyn, et al., J. Immunol. Meth. 73:157-167, 1984), cell (e.g., CTL, helper T-cell) or humoral (e.g., antibody) mediated inhibition of cell growth in vitro (Gazit, et al., Cancer Immunol Immunother 35:135-144, 1992), and, for any of these assays, determination of cell precursor frequency (Vose, Int. J. Cancer 30:135-142 (1982), and others.

Methods of determining the presence and magnitude of an immune response are well known in the art. In one embodiment, lymphocyte proliferation assays, wherein T cell uptake of a radioactive substance, e.g. ³H-thymidine is measured as a function of cell proliferation. In other embodiments, detection of T cell proliferation is accomplished by measuring increases in interleukin-2 (IL-2) production, Ca²⁺ flux, or dye uptake, such as 3-(4,5-dimethylthiazol-2-yl)-2,5-diphenyl-tetrazolium. Each possibility represents a separate embodiment of the present invention

In another embodiment, CTL stimulation is determined by means known to those skilled in the art, including, detection of cell proliferation, cytokine production and others. Analysis of the types and quantities of cytokines secreted by T cells upon contacting ligand-pulsed targets can be a measure of functional activity. Cytokines can be measured by ELISA or ELISPOT assays to determine the rate and total amount of cytokine production. (Fujihashi K. et al. (1993) J. Immunol. Meth. 160:181; Tanguay S. and Killion J. J. (1994) Lymphokine Cytokine Res. 13:259).

In another embodiment, CTL activity is determined by ⁵¹Cr-release lysis assay. Lysis of peptide-pulsed ⁵¹Cr-labeled targets by antigen-specific T cells can be compared for target cells pulsed with control peptide. In another embodiment, T cells are stimulated with a peptide of this invention, and lysis of target cells expressing the native peptide in the context of MHC can be determined. The kinetics of lysis as well as overall target lysis at a fixed timepoint (e.g., 4 hours) are used, in another embodiment, to evaluate ligand performance. (Ware C. F. et al. (1983) J. Immunol. 131:1312).

Methods of determining affinity of a peptide for an HLA molecule are well known in the art. In one embodiment, affinity is determined by TAP stabilization assays (Examples).

In another embodiment, affinity is determined by competition radioimmunoassay. In one embodiment, the following protocol is utilized: Target cells are washed two times in PBS with 1% bovine serum albumin (BSA; Fisher Chemicals, Fairlawn, N.J.). Cells are resuspended at 10⁷/ml on ice, and the native cell surface bound peptides are stripped for 2 minutes at 0° C. using citrate-phosphate buffer in the presence of 3 mg/ml beta₂ microglobulin. The pellet is resuspended at 5×10⁶ cells/ml in PBS/1% BSA in the presence of 3 mg/ml beta₂ microglobulin and 30 mg/ml deoxyribonuclease, and 200 ml aliquots are incubated in the presence or absence of HLA-specific peptides for 10 min at 20° C., then with ¹²⁵I-labeled peptide for 30 min at 20° C. Total bound ¹²⁵I is determined after two washes with PBS/2% BSA and one wash with PBS. Relative affinities are determined by comparison of escalating concentrations of the test peptide versus a known binding peptide.

In another embodiment, a specificity analysis of the binding of peptide to HLA on surface of live cells (e.g. SKLY-16 cells) is conducted to show that binding is to the appropriate HLA molecule and to characterize its restriction. This includes, in another embodiment, competition with excess unlabeled peptides known to bind to the same or disparate HLA molecules and use of target cells which express the same or disparate HLA types. This assay is performed, in one embodiment, on live fresh or 0.25% paraformaldehyde-fixed human PBMC, leukemia cell lines and EBV-transformed T-cell lines of specific HLA types. The relative avidity of the peptides found to bind MHC molecules on the specific cells are assayed by competition assays as described above against ¹²⁵I-labeled peptides of known high affinity for the relevant HLA molecule, e,g., tyrosinase or HBV peptide sequence

In another embodiment, a vaccine of the present invention comprises an unmutated bcr-abl vaccine peptide that binds an HLA class II molecule and a mutant bcr-abl vaccine peptide that binds an HLA class I molecule. In one embodiment, inclusion of HLA class I-binding and HLA class I-binding peptides in the same vaccine enables synergistic activation of the anti-bcr-abl immune response by activating CD4⁺ and CD8⁺ T cells that recognize the same target. Each possibility represents a separate embodiment of the present invention

In another embodiment, the HLA class II-binding peptide is longer than the minimum length for binding to an HLA class II molecule, which is, in one embodiment, about 12 AA. In another embodiment, increasing the length of the HLA class II-binding peptide enables binding to more than one HLA class II molecule. In another embodiment, increasing the length enables binding to an HLA class II molecule whose binding motif is not known. In another embodiment, increasing the length enables binding to an HLA class I molecule. In one embodiment, the binding motif of the HLA class I molecule is known. In another embodiment, the binding motif of the HLA class I molecule is not known. Each possibility represents a separate embodiment of the present invention.

Methods for predicting MHC class II epitopes are well known in the art. In one embodiment, the MHC class II epitope is predicted using TEPITOPE (Meister G E, Roberts C G et al, Vaccine 1995 13: 581-91) In another embodiment, the MHC class II epitope is predicted using EpiMatrix (De Groot A S, Jesdale B M et al, AIDS Res. Hum. Retroviruses 1997 13: 529-31). In another embodiment, the MHC class II epitope is predicted using the Predict Method (Yu K, Petrovsky N et al, Mol Med. 2002 8:137-48). In another embodiment, the MHC class II epitope is predicted using any other method known in the art. Each possibility represents a separate embodiment of the present invention.

In another embodiment, the peptides utilized in methods and compositions of the present invention comprise a non-classical amino acid such as: 1,2,3,4-tetrahydroisoquinoline-3-carboxylate (Kazmierski et al. (1991) J. Am Chem. Soc. 113:2275-2283); (2S,3S)-methyl-phenylalanine, (2S,3R)-methyl-phenylalanine, (2R,3S)-methyl-phenylalanine and (2R,3R)-methyl-phenylalanine (Kazmierski and Hruby (1991) Tetrahedron Lett. 32(41): 5769-5772); 2-aminotetrahydronaphthalene-2-carboxylic acid (Landis (1989) Ph.D. Thesis, University of Arizona); hydroxy-1,2,3,4-tetrahydroisoquinoline-3-carboxylate (Miyake et al. (1984) J. Takeda Res. Labs. 43:53-76) histidine isoquinoline carboxylic acid (Zechel et al. (1991) Int. J. Pep. Protein Res. 38(2):131-138); and HIC (histidine cyclic urea), (Dharanipragada et al. (11993) Int. J. Pep. Protein Res. 42(1):68-77) and ((1992) Acta. Crst., Crystal Struc. Comm. 48(IV): 1239-124).

In another embodiment, a peptide of this invention comprises an AA analog or peptidomimetic, which, in other embodiments, induces or favors specific secondary structures. Such peptides comprise, in other embodiments, the following: LL-Acp (LL-3-amino-2-propenidone-6-carboxylic acid), a β-turn inducing dipeptide analog (Kemp et al (1985) J. Org. Chem. 50:5834-5838); β-sheet inducing analogs (Kemp et al. (1988) Tetrahedron Lett. 29:5081-5082); β-turn inducing analogs (Kemp et al. (1988) Tetrahedron Left. 29:5057-5060); .alpha.-helix inducing analogs (Kemp et al. (1988) Tetrahedron Left. 29:4935-4938); gamma.-turn inducing analogs (Kemp et al. (1989) J. Org. Chem. 54:109:115); analogs provided by the following references: Nagai and Sato (1985) Tetrahedron Left. 26:647-650; and DiMaio et al. (1989) J. Chem. Soc. Perkin Trans. p. 1687; a Gly-Ala turn analog (Kahn et al. (1989) Tetrahedron Lett. 30:2317); amide bond isostere (Jones et al. (1988) Tetrahedron Left. 29(31):3853-3856); tretrazol (Zabrocki et al. (1988) J. Am. Chem. Soc. 110:5875-5880); DTC (Samanen et al. (1990) Int. J. Protein Pep. Res. 35:501:509); and analogs taught in Olson et al. (1990) J. Am, Chem. Sci. 112:323-333 and Garveyet al. (1990) J. Org. Chem. 55(3):936-940. Conformationally restricted mimetics of beta turns and beta bulges, and peptides containing them, are described in U.S. Pat. No. 5,440,013, issued Aug. 8, 1995 to Kahn.

In other embodiments, a peptide of this invention is conjugated to one of various other molecules, as described hereinbelow, which can be via covalent or non-covalent linkage (complexed), the nature of which varies, in another embodiment, depending on the particular purpose. In another embodiment, the peptide is covalently or non-covalently complexed to a macromolecular carrier, (e.g. an immunogenic carrier), including, but not limited to, natural and synthetic polymers, proteins, polysaccharides, polypeptides (amino acids), polyvinyl alcohol, polyvinyl pyrrolidone, and lipids. In another embodiment, a peptide of this invention is linked to a substrate. In another embodiment, the peptide is conjugated to a fatty acid, for introduction into a liposome (U.S. Pat. No. 5,837,249). In another embodiment, a peptide of the invention is complexed covalently or non-covalently with a solid support, a variety of which are known in the art. In another embodiment, linkage of the peptide to the carrier, substrate, fatty acid, or solid support serves to increase an elicited an immune response

In other embodiments, the carrier is thyroglobulin, an albumin (e.g. human serum albumin), tetanus toxoid, polyamino acids such as poly (lysine: glutamic acid), an influenza protein, hepatitis B virus core protein, keyhole limpet hemocyanin, an albumin, or another carrier protein or carrier peptide; hepatitis B virus recombinant vaccine, or an APC. Each possibility represents a separate embodiment of the present invention.

In another embodiment, the term “amino acid” refers to a natural or, in another embodiment, an unnatural or synthetic AA, and can include, in other embodiments, glycine, D- or L optical isomers, AA analogs, peptidomimetics, or combinations thereof.

In another embodiment, the terms “cancer,” “neoplasm,” “neoplastic” or “tumor,” are used interchangeably and refer to cells that have undergone a malignant transformation that makes them pathological to the host organism. Primary cancer cells (that is, cells obtained from near the site of malignant transformation) can be readily distinguished from non-cancerous cells by well-established techniques, particularly histological examination. The definition of a cancer cell, as used herein, includes not only a primary cancer cell, but also any cell derived from a cancer cell ancestor. This includes metastasized cancer cells, and in vitro cultures and cell lines derived from cancer cells. In one embodiment, a tumor is detectable on the basis of tumor mass; e.g., by such procedures as CAT scan, magnetic resonance imaging (MRI), X-ray, ultrasound or palpation, and in another embodiment, is identified by biochemical or immunologic findings, the latter which is used to identify cancerous cells, as well, in other embodiments.

Methods for synthesizing peptides are well known in the art. In one embodiment, the peptides of this invention are synthesized using an appropriate solid-state synthetic procedure (see for example, Steward and Young, Solid Phase Peptide Synthesis, Freemantle, San Francisco, Calif. (1968); Merrifield (1967) Recent Progress in Hormone Res 23: 451). The activity of these peptides is tested, in other embodiments, using assays as described herein.

In another embodiment, the peptides of this invention are purified by standard methods including chromatography (e.g., ion exchange, affinity, and sizing column chromatography), centrifugation, differential solubility, or by any other standard technique for protein purification. In another embodiment, immuno-affinity chromatography is used, whereby an epitope is isolated by binding it to an affinity column comprising antibodies that were raised against that peptide, or a related peptide of the invention, and were affixed to a stationary support.

In another embodiment, affinity tags such as hexa-His (Invitrogen), Maltose binding domain (New England Biolabs), influenza coat sequence (Kolodziej et al. (1991) Meth. Enzymol. 194:508-509), glutathione-S-transferase, or others, are attached to the peptides of this invention to allow easy purification by passage over an appropriate affinity column. Isolated peptides can also be physically characterized, in other embodiments, using such techniques as proteolysis, nuclear magnetic resonance, and x-ray crystallography.

In another embodiment, the peptides of this invention are produced by in in vitro translation, through known techniques, as will be evident to one skilled in the art. In another embodiment, the peptides are differentially modified during or after translation, e.g., by phosphorylation, glycosylation, cross-linking, acylation, proteolytic cleavage, linkage to an antibody molecule, membrane molecule or other ligand, (Ferguson et al. (1988) Ann. Rev. Biochem. 57:285-320).

In one embodiment, the peptides of this invention further comprise a detectable label, which in one embodiment, is fluorescent, or in another embodiment, luminescent, or in another embodiment, radioactive, or in another embodiment, electron dense. In other embodiments, the dectectable label comprises, for example, green fluorescent protein (GFP), DS-Red (red fluorescent protein), secreted alkaline phosphatase (SEAP), beta-galactosidase, luciferase, ³²P, ¹²⁵I, ³H and ¹⁴C, fluorescein and its derivatives, rhodamine and its derivatives, dansyl and umbelliferone, luciferin or any number of other such labels known to one skilled in the art. The particular label used will depend upon the type of immunoassay used.

In another embodiment, a peptide of this invention is linked to a substrate, which, in one embodiment, serves as a carrier. In one embodiment, linkage of the peptide to a substrate serves to increase an elicited an immune response.

In one embodiment, peptides of this invention are linked to other molecules, as described herein, using conventional cross-linking agents such as carbodimides. Examples of carbodimides are 1-cyclohexyl-3-(2-morpholinyl-(4-ethyl) carbodiimide (CMC), 1-ethyl-3-(3-dimethyaminopropyl) carbodiimide (EDC) and 1-ethyl-3-(4-azonia-44-dimethylpentyl) carbodiimide.

In other embodiments, the cross-linking agents comprise cyanogen bromide, glutaraldehyde and succinic anhydride. In general, any of a number of homo-bifunctional agents including a homo-bifunctional aldehyde, a homo-bifunctional epoxide, a homo-bifunctional imido-ester, a homo-bifunctional N-hydroxysuccinimide ester, a homo-bifunctional maleimide, a homo-bifunctional alkyl halide, a homo-bifunctional pyridyl disulfide, a homo-bifunctional aryl halide, a homo-bifunctional hydrazide, a homo-bifunctional diazonium derivative and a homo-bifunctional photoreactive compound can be used. Also envisioned, in other embodiments, are hetero-bifunctional compounds, for example, compounds having an amine-reactive and a sulfhydryl-reactive group, compounds with an amine-reactive and a photoreactive group and compounds with a carbonyl-reactive and a sulfhydryl-reactive group

In other embodiments, the homo-bifunctional cross-linking agents include the bifunctional N-hydroxysuccinimide esters dithiobis(succinimidylpropionate), disuccinimidyl suberate, and disuccinimidyl tartarate; the bifunctional imido-esters dimethyl adipimidate, dimethyl pimelimidate, and dimethyl suberimidate; the bifunctional sulfhydryl-reactive crosslinkers 1,4-di-[3′-(2′-pyridyldithio)propionamido]butane, bismaleimidohexane, and bis-N-maleimido-1,8-octane; the bifunctional aryl halides 1,5-difluoro-2,4-dinitrobenzene and 4,4′-difluoro-3,3′-dinitrophenylsulfone; bifunctional photoreactive agents such as bis-[b-(4-azidosalicylamido)ethyl]disulfide; the bifunctional aldehydes formaldehyde, malondialdehyde, succinaldehyde, glutaraldehyde, and adipaldehyde; a bifunctional epoxide such as 1,4-butaneodiol diglycidyl ether; the bifunctional hydrazides adipic acid dihydrazide, carbohydrazide, and succinic acid dihydrazide; the bifunctional diazoniums o-tolidine, diazotized and bis-diazotized benzidine; the bifunctional alkylhalides N1N′-ethylene-bis(iodoacetamide), N1N′-hexamethylene-bis(iodoacetamide), N1N′-undecamethylene-bis(iodoacetamide), as well as benzylhalides and halomustards, such as ala′-diiodo-p-xylene sulfonic acid and tri(2-chloroethyl)amine, respectively.

In other embodiments, hetero-bifunctional cross-linking agents used to link the peptides to other molecules, as described herein, include, but are not limited to, SMCC (succinimidyl-4-(N-maleimidomethyl)cyclohexane-1-carboxylate), MBS (m-maleimidobenzoyl-N-hydroxysuccinimide ester), SMPB (N-succinimidyl(4-iodoacteyl)aminobenzoate), SMPB (succinimidyl-4-(p-maleimidophenyl)butyrate), GMBS (N-(.gamma.-maleimidobutyryloxy)succinimide ester), MPBH (4-(4-N-maleimidopohenyl) butyric acid hydrazide), M2C2H (4-(N-maleimidomethyl) cyclohexane-1-carboxyl-hydrazide), SMPT (succinimidyloxycarbonyl-a-methyl-a-(2-pyridyldithio)toluene), and SPDP (N-succinimidyl 3-(2-pyridyldithio)propionate).

In another embodiment, the peptides of the invention are formulated as non-covalent attachment of monomers through ionic, adsorptive, or biospecific interactions. Complexes of peptides with highly positively or negatively charged molecules can be accomplished, in another embodiment, through salt bridge formation under low ionic strength environments, such as in deionized water Large complexes can be created, in another embodiment, using charged polymers such as poly-(L-glutamic acid) or poly-(L-lysine), which contain numerous negative and positive charges, respectively. In another embodiment, peptides are adsorbed to surfaces such as microparticle latex beads or to other hydrophobic polymers, forming non-covalently associated peptide-superantigen complexes effectively mimicking cross-linked or chemically polymerized protein, in other embodiments. In another embodiment, peptides are non-covalently linked through the use of biospecific interactions between other molecules. For instance, utilization of the strong affinity of biotin for proteins such as avidin or streptavidin or their derivatives could be used to form peptide complexes. The peptides, according to this aspect, and in one embodiment, can be modified to possess biotin groups using common biotinylation reagents such as the N-hydroxysuccinimidyl ester of D-biotin (NHS-biotin), which reacts with available amine groups.

In another embodiment, the peptides are linked to carriers. In another embodiments, the peptides are any that are well known in the art, including, for example, thyroglobulin, albumins such as human serum albumin, tetanus toxoid, polyamino acids such as poly (lysine:glutamic acid), influenza, hepatitis B virus core protein, hepatitis B virus recombinant vaccine and the like. Each possibility represents a separate embodiment of the present invention.

In another embodiment, the peptides of this invention are conjugated to a lipid, such as P3 CSS. In another embodiment, the peptides of this invention are conjugated to a bead.

In another embodiment, the compositions of this invention further comprise immunomodulating compounds. In other embodiments, the immunomodulating compound is a cytokine, chemokine, or complement component that enhances expression of immune system accessory or adhesion molecules, their receptors, or combinations thereof. In some embodiments, the immunomodulating compound include interleukins, for example interleukins 1 to 15, interferons alpha, beta or gamma, tumour necrosis factor, granulocyte-macrophage colony stimulating factor (GM-CSF), macrophage colony stimulating factor (M-CSF), granulocyte colony stimulating factor (G-CSF), chemokines such as neutrophil activating protein (NAP), macrophage chemoattractant and activating factor (MCAF), RANTES, macrophage inflammatory peptides MIP-1a and MIP-1b, complement components, or combinations thereof. In other embodiments, the immunomodulating compound stimulate expression, or enhanced expression of OX40, OX40L (gp34), lymphotactin, CD40, CD40L, B7.1, B7.2, TRAP, ICAM-1, 2 or 3, cytokine receptors, or combination thereof.

In another embodiment, the immunomodulatory compound induces or enhances expression of co-stimulatory molecules that participate in the immune response, which include, in some embodiments, CD40 or its ligand, CD28, CTLA4 or a B7 molecule. In another embodiment, the immunomodulatory compound induces or enhances expression of a heat stable antigen (HSA) (Liu Y. et al. (1992) J. Exp. Med. 175:437-445), chondroitin sulfate-modified MHC invariant chain (Ii-CS) (Naujokas M. F. et al. (1993) Cell 74:257-268), or an intracellular adhesion molecule 1 (ICAM-1) (Van R. H. (1.992) Cell 71:1065-1068), which assists, in another embodiment, co-stimulation by interacting with their cognate ligands on the T cells.

In another embodiment, the composition comprises a solvent, including water, dispersion media, cell culture media, isotonic agents and the like. In one embodiment, the solvent is an aqueous isotonic buffered solution with a pH of around 7.0. In another embodiment, the composition comprises a diluent such as water, phosphate buffered saline, or saline. In another embodiment, the composition comprises a solvent, which is non-aqueous, such as propyl ethylene glycol, polyethylene glycol and vegetable oils.

In another embodiment, the composition is formulated for administration by any of the many techniques known to those of skill in the art. For example, this invention provides for administration of the pharmaceutical composition parenterally, intravenously, subcutaneously, intradermally, intramucosally, topically, orally, or by inhalation.

In another embodiment, the vaccine comprising a peptide of this invention further comprises a cell population, which, in another embodiment, comprises lymphocytes, monocytes, macrophages, dendritic cells, endothelial cells, stem cells or combinations thereof, which, in another embodiment are autologous, syngeneic or allogeneic, with respect to each other. In another embodiment, the cell population comprises a peptide of the present invention. In another embodiment, the cell population takes up the peptide. Each possibility represents a separate embodiment of the present invention.

In one embodiment, the cell populations of this invention are obtained from in vivo sources, such as, for example, peripheral blood, leukoplieresis blood product, apheresis blood product, peripheral lymph nodes, gut associated lymphoid tissue, spleen, thymus, cord blood, mesenteric lymph nodes, liver, sites of immunologic lesions, e.g. synovial fluid, pancreas, cerebrospinal fluid, tumor samples, granulomatous tissue, or any other source where such cells can be obtained. In one embodiment, the cell populations are obtained from human sources, which are, in other embodiments, from human fetal, neonatal, child, or adult sources. In another embodiment, the cell populations of this invention are obtained from animal sources, such as, for example, porcine or simian, or any other animal of interest. In another embodiment, the cell populations of this invention are obtained from subjects that are normal, or in another embodiment, diseased, or in another embodiment, susceptible to a disease of interest.

In another embodiment, the cell populations of this invention are separated via affinity-based separation methods. Techniques for affinity separation include, in other embodiments, magnetic separation, using antibody-coated magnetic beads, affinity chromatography, cytotoxic agents joined to a monoclonal antibody or use in conjunction with a monoclonal antibody, for example, complement and cytotoxins, and “panning” with an antibody attached to a solid matrix, such as a plate, or any other convenient technique. In other embodiment, separation techniques include the use of fluorescence activated cell sorters, which can have varying degrees of sophistication, such as multiple color channels, low angle and obtuse light scattering detecting channels, impedance channels, etc. In other embodiments, any technique that enables separation of the cell populations of this invention can be employed, and is to be considered as part of this invention.

In one embodiment, the dendritic cells are from the diverse population of morphologically similar cell types found in a variety of lymphoid and non-lymphoid tissues, qualified as such (Steinman (1991) Ann. Rev Immunol. 9:271-296). In one embodiment, the dendritic cells used in this invention are isolated from bone marrow, or in another embodiment, derived from bone marrow progenitor cells, or, in another embodiment, from isolated from/derived from peripheral blood, or in another embodiment, derived from, or are a cell line.

In one embodiment, the cell populations described herein are isolated from the white blood cell fraction of a mammal, such as a murine, simian or a human (See, e.g., WO 96/23060). The white blood cell fraction can be, in another embodiment, isolated from the peripheral blood of the mammal.

Methods of isolating dendritic cells are well known in the art. In one embodiment, the DC are isolated via a method which includes the following steps: (a) providing a white blood cell fraction obtained from a mammalian source by methods known in the art such as leukophoresis; (b) separating the white blood cell fraction of step (a) into four or more subfractions by countercurrent centrifugal elutriation; (c) stimulating conversion of monocytes in one or more fractions from step (b) to dendritic cells by contacting the cells with calcium ionophore, GM-CSF and IL-13 or GM-CSF and IL-4, (d) identifying the dendritic cell-enriched fraction from step (c); and (e) collecting the enriched fraction of step (d), preferably at about 4° C.

In another embodiment, the dendritic cell-enriched fraction is identified by fluorescence-activated cell sorting, which identifies at least one of the following markers: HLA-DR, HLA-DQ, or B7.2, and the simultaneous absence of the following markers: CD3, CD14, CD16, 56, 57, and CD 19, 20.

In another embodiment, the cell population comprises lymphocytes, which are, in one embodiment, T cells, or in another embodiment, B cells. The T cells are, in other embodiments, characterized as NK cells, helper T cells, cytotoxic T lymphocytes (CTL), TILs, naïve T cells, or combinations thereof. It is to be understood that T cells which are primary, or cell lines, clones, etc. are to be considered as part of this invention. In one embodiment, the T cells are CTL, or CTL lines, CTL clones, or CTLs isolated from tumor, inflammatory, or other infiltrates.

In another embodiment, hematopoietic stem or early progenitor cells comprise the cell populations used in this invention. In one embodiment, such populations are isolate or derived, by leukaphoresis. In another embodiment, the leukaphoresis follows cytokine administration, from bone marrow, peripheral blood (PB) or neonatal umbilical cord blood. In one embodiment the stem or progenitor cells are characterized by their surface expression of the surface antigen marker known as CD34+, and exclusion of expression of the surface lineage antigen markers, Lin-.

In another embodiment, the subject is administered a peptide, composition or vaccine of this invention, in conjunction with bone marrow cells. In another embodiment, the administration together with bone marrow cells embodiment follows previous irradiation of the subject, as part of the course of therapy, in order to suppress, inhibit or treat cancer in the subject.

In one embodiment, the phrase “contacting a cell” or “contacting a population” refers to a method of exposure, which can be, in other embodiments, direct or indirect. In another embodiment, such contact comprises direct injection of the cell through any means well known in the art, such as microinjection. It is also envisaged, in another embodiment, that supply to the cell is indirect, such as via provision in a culture medium that surrounds the cell, or administration to a subject, via any route well known in the art, and as described herein.

In one embodiment, CTL generation of methods of the present invention is accomplished in vivo, and is effected by introducing into a subject an antigen presenting cell contacted in vitro with a peptide of this invention (See for example Paglia et al. (1996) J. Exp. Med. 183:317-322).

In another embodiment, the peptides of methods and compositions of the present invention are delivered to antigen-presenting cells (APC).

In another embodiment, the peptides are delivered to APC in the form of cDNA encoding the peptides. In one embodiment, the term “antigen-presenting cells” refers to dendritic cells (DC), monocytes/macrophages, B lymphocytes or other cell type(s) expressing the necessary MHC/co-stimulatory molecules, which effectively allow for T cell recognition of the presented peptide. In another embodiment, the APC is a cancer cell. Each possibility represents a separate embodiment of the present invention.

In another embodiment, the CTL are contacted with two or more antigen-presenting cell populations In another embodiment, the two or more antigen presenting cell populations present different peptides Each possibility represents a separate embodiment of the present invention

In another embodiment, techniques that lead to the expression of antigen in the cytosol of APC (e.g. DC) are used to deliver the peptides to the APC Methods for expressing antigens on APC are well known in the art In one embodiment, the techniques include (1) the introduction into the APC of naked DNA encoding a peptide of this inveniton, (2) infection of APC with recombinant vectors expressing a peptide of this invention, and (3) introduction of a peptide of this invention into the cytosol of an APC using liposomes. (See Boczkowski D. et al. (1996) J. Exp. Med. 184:465-472; Rouse et al. (1994) J. Virol. 68:5685-5689; and Nair et al. (1992) J. Exp. Med. 175:609-612).

In another embodiment, foster antigen presenting cells such as those derived from the human cell line 174xCEM.T2, referred to as T2, which contains a mutation in its antigen processing pathway that restricts the association of endogenous peptides with cell surface MHC class I molecules (Zweerink et al. (1993) J. Immunol 150:1763-1771), are used, as exemplified herein.

In one embodiment, as described herein, the subject is exposed to a peptide, or a composition/cell population comprising a peptide of this invention, which differs from the native protein expressed, wherein subsequently a host immune cross-reactive with the native protein/antigen develops

In one embodiment, the subject, as referred to in any of the methods or embodiments of this invention is a human. In other embodiments, the subject is a mammal, which can be a mouse, rat, rabbit, hamster, guinea pig, horse, cow, sheep, goat, pig, cat, dog, monkey, or ape. Each possibility represents a separate embodiment of the present invention.

In another embodiment, peptides, vaccines, and compositions of this invention stimulate an immune response that results in tumor cell lysis.

In one embodiment, any of the methods described herein is used to elicit CTL, which are elicited in vitro. In another embodiment, the CTL are elicited ex-vivo. In another embodiment, the CTL are elicited in vitro. The resulting CTL, are, in another embodiment, administered to the subject, thereby treating the condition associated with the peptide, an expression product comprising the peptide, or a homologue thereof. Each possibility represents a separate embodiment of the present invention.

In another embodiment, the method entails introduction of the genetic sequence that encodes the peptides of this invention. In one embodiment, the method comprises administering to the subject a vector comprising a nucleotide sequence, which encodes a peptide of the present invention (Tindle, R. W. et al. Virology (1994) 200:54). In another embodiment, the method comprises administering to the subject naked DNA which encodes a peptide, or in another embodiment, two or more peptides of this invention (Nabel, et al. PNAS-USA (1990) 90: 11307). In another embodiment, multi-epitope, analogue-based cancer vaccines are utilized (Fikes et al, ibid). Each possibility represents a separate embodiment of the present invention.

Nucleic acids can be administered to a subject via any means as is known in the art, including parenteral or intravenous administration, or in another embodiment, by means of a gene gun. In another embodiment, the nucleic acids are administered in a composition, which correspond, in other embodiments, to any embodiment listed herein.

Vectors for use according to methods of this invention can comprise any vector that facilitates or allows for the expression of a peptide of this invention. Vectors comprises, in some embodiments, attenuated viruses, such as vaccinia or fowlpox, such as described in, e.g., U.S. Pat. No. 4,722,848, incorporated herein by reference. In another embodiment, the vector is BCG (Bacille Calmette Guerin), such as described in Stover et al. (Nature 351:456-460 (1991)). A wide variety of other vectors useful for therapeutic administration or immunization of the peptides of the invention, e.g., Salmonella typhi vectors and the like, will be apparent to those skilled in the art from the description herein.

In one embodiment, the vector further encodes for an immunomodulatory compound, as described herein. In another embodiment, the subject is administered an additional vector encoding same, concurrent, prior to or following administration of the vector encoding a peptide of this invention to the subject.

In another embodiment, the subject is administered a peptide following previous administration of chemotherapy to the subject. In another embodiment, the subject has been treated with imatinib. In another embodiment, the cancer in the subject is resistant to imatinib treatment.

In another embodiment, methods of suppressing tumor growth indicate a growth state that is curtailed compared to growth without contact with, or exposure to a peptide of this invention. Tumor cell growth can be assessed by any means known in the art, including, but not limited to, measuring tumor size, determining whether tumor cells are proliferating using a ³H-thymidine incorporation assay, or counting tumor cells. “Suppressing” tumor cell growth refers, in other embodiments, to slowing, delaying, or stopping tumor growth, or to tumor shrinkage Each possibility represents a separate embodiment of the present invention.

In another embodiment, the peptides, compositions and vaccines of this invention are administered to a subject, or utilized in the methods of this invention, in combination with other anti-cancer compounds and chemotherapeutics, including monoclonal antibodies directed against alternate cancer antigens, or, in another embodiment, epitopes that consist of an AA sequence which corresponds to, or in part to, that from which the peptides of this invention are derived.

Various embodiments of dosage ranges are contemplated by this invention In one embodiment, the dosage is 20 μg per peptide per day. In another embodiment, the dosage is 10 μg mg/peptide/day. In another embodiment, the dosage is 30 μg mg/peptide/day. In another embodiment, the dosage is 40 μg mg/peptide/day. In another embodiment, the dosage is 60 μg mg/peptide/day. In another embodiment, the dosage is 80 μg mg/peptide/day. In another embodiment, the dosage is 100 μg mg/peptide/day. In another embodiment, the dosage is 150 μg mg/peptide/day. In another embodiment, the dosage is 200 μg mg/peptide/day.

In another embodiment, the dosage is 10 μg mg/peptide/dose. In another embodiment, the dosage is 30 μg mg/peptide/dose. In another embodiment, the dosage is 40 μg mg/peptide/dose. In another embodiment, the dosage is 60 μg mg/peptide/dose. In another embodiment, the dosage is 80 μg mg/peptide/dose. In another embodiment, the dosage is 100 μg mg/peptide/dose. In another embodiment, the dosage is 150 μg mg/peptide/dose. In another embodiment, the dosage is 200 μg mg/peptide/dose.

In another embodiment, the total peptide dose per day is one of the above amounts. In another embodiment, the total peptide dose per dose is one of the above amounts.

Each of the above doses represents a separate embodiment of the present invention.

### EXPERIMENTAL DETAILS SECTION

### Example 1

### Proliferation of T Cells in Response to Stimulation with Wild-Type bcr-abl Breakpoint Peptides

### Materials and Experimental Methods

Peptide Stimulations

PBMC were purified by centrifugation in Ficoll-Paque centrifugation medium (Amersham Biosciences), then were depleted of CD4⁺ cells by using anti-CD4 antibody-coated magnetic beads (Dynabeads®, Oslo). Non-transformed lymphoblasts were used as APC, and were prepared by incubating 2×10ˆ6/ml PBMC with 0.005% (vol/vol) Staphylococcus Aureus Cowan-I (Pansorbin, Calbiochem), 20 μg/ml rabbit anti-human IgM antibody coupled to Immunobeads® (Bio-Rad), and recombinant interleukin 4 (IL-4; Sandoz Pharmaceutical) in Gibco RPMI 1640 (Invitrogen) in 24-well tissue culture plates. Lymphoblasts were then incubated overnight at 26° C., 5% CO₂, loaded with 50 μg/ml peptide in the presence of 3 μg/ml human β₂ microglobulin for 4 hours (h), 20° C. in phosphate-buffered saline (PBS), and gamma-irradiated with 6000 rads (1 rad=0.04 Gy). 10ˆ6 of the resulting cells were mixed at a ratio of 1:3 with autologous CD4⁺ cell-depleted PMBC and incubated in RPMI 1640+5% heat-inactivated AB human serum and recombinant 10 ng/ml IL-7 (Genzyme), for 12 days (d) at 37° C., 5% CO₂. Recombinant IL-2 (Sandoz Pharmaceutical) was added to the cultures during days 12-14, The CD4⁺ cell-depleted PMBC were re-stimulated every 7-10 d, at 10ˆ6 cells per well, with peptide-incubated autologous irradiated adherent cells. Irradiated adherent cells were prepared by incubating 4×10ˆ6 irradiated (3500 rad) PMBC in 0.5 ml medium for 2 h, 37° C., to a 24-well tissue culture plate, removing non-adherent cells, and incubating the remaining cells with 10 μg/ml peptide and 3 μg/ml human β₂ microglobulin in 0.5 ml for 2 h. After removing excess peptide, the irradiated adherent cells were incubated with the CD4⁺ cell-depleted PMBC, adding fresh IL-2-containing media every 3-5 days.

### Results

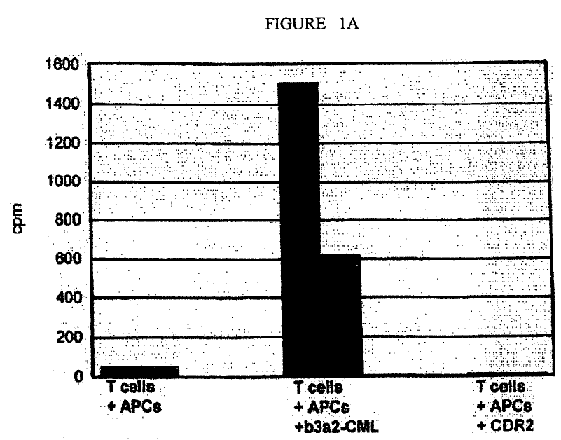

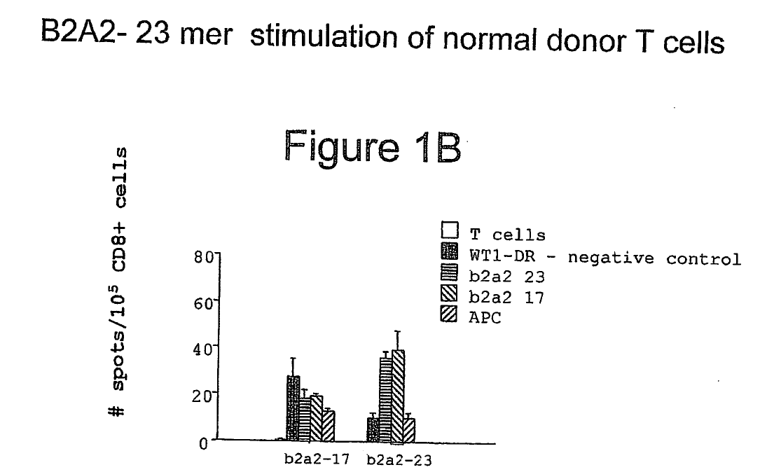

To test the immunogenicity of a 25 amino acid (AA) b3a2-bcr-abl derived peptide, IVHSATGFKQSSKALQRPVASDFEP (SEQ ID No: 24), Peripheral Blood Mononuclear Cells (PBMC) from a healthy donor were stimulated with irradiated or paraformaldehyde-fixed (negative control) autologous PBMC pulsed with the b3a2 peptide, or with no peptide or an irrelevant peptide (additional negative controls). After two sets of stimulations on day 0 and day 12, T cells were incubated on day 19 with autologous PBMC, used as APC (1:1 ratio) that were either not peptide-pulsed, pulsed with b3a2-CML peptide, or pulsed with a 17 AA control peptide (CDR2). After 72 hours of culture, specific proliferation was measured by ³H-thymidine incorporation. The PBMC pulsed with b3a2 peptide, but not the other 2 groups, induced proliferation of antigen-specific T cells (FIG. 1).

Next the immunogenicity of a 23 AA b2a2-bcr-abl derived peptide, (VHSIPLTINKEEALQRPVASDFE; SEQ ID No: 17), was tested. CD3⁺ cells were stimulated twice with the 23 AA peptide or a 17 AA fragment thereof (IPLTINKEEALQRPVAS, SEQ ID No: 20), then IFN-γ production in response to each of these peptides or WT1-DR (negative control) was assayed by ELISPOT. Stimulation with the 23. AA peptide induced T cells that recognized both the 23 AA and 17 AA peptide. By contrast, cells stimulated with the 17 AA fragment did not secrete greater than background levels of IFN-γ. Similar results were obtained with total PBMC.

### Example 2

### Identification and Generation of Peptides with a High Probability of HLA A0201 Binding

### Materials and Experimental Methods

Peptides

Peptides were synthesized by Genemed Synthesis Inc, CA using fluorenylmethoxycarbonyl chemistry and solid phase synthesis, and were purified by high pressure liquid chromatography (HPLC). The quality of the peptides was assessed by HPLC analysis, and the expected molecular weight was measured using matrix-assisted laser desorption mass spectrometry. Peptides were sterile and >90% pure. The peptides were dissolved in DMSO and diluted in PBS at pH 7.4 or saline solution to yield a concentration of 5 milligrams per milliliter (mg/ml) and were stored at −80° C. For in vitro experiments, an irrelevant control peptide, HLA A24 consensus, was used.

Peptide Sequence Analysis

Peptide sequence analysis was performed using 2 databases. The first was the software of the Bioinformatics & Molecular Analysis Section (National Institutes of Health, Washington, D.C.) (Parker K C et al, Scheme for ranking potential HLA-A2 binding peptides based on independent binding of individual peptide side-chains. J. Immunol 152: 163-175, 1994), which ranks 9-mer or 10-mer peptides on a predicted half-time dissociation coefficient from HLA class I molecules. The second database, SYFPEITHI prediction software, is described in Rammensee H G et al (SYFPEITHI: database for MHC ligands and peptide motifs. Immunogenetics 50: 213-219, 1999).

### Results

Peptides with potential CTL epitopes were predicted by means of a peptide library-based scoring system for MHC class I-binding peptides Junctional (“breakpoint”) amino acid sequences of the human b3a2 and b2a2 fusion proteins were scanned for peptides with potential binding capacity for HLA A0201, a subtype encompassing 95% of the HLA-A02 allele. HLA-A0201 is expressed in about 40% of the Caucasian population. No peptides with high or intermediate affinity, defined as having a predicted half life of greater than 1 minute, were identified in the native b3a2 or b2a2 fusion proteins.

Using the software of the Bioinformatics & Molecular Analysis Section, several analogue peptides of bcr-abl b3a2 and b2a2 breakpoint peptides were designed, wherein one or both anchor amino acids or additional amino acids adjacent to anchor amino acids were modified. Single or double AA substitutions were introduced at HLA A0201 preferred residues (positions 1, 2, 6 and 9, see underlined residues in Table 1) to yield sequences that had comparatively high binding scores predicted for HLA A0201 molecules. The predicted half life for binding to HLA A0201 was greater than 240 minutes in four synthetic peptides and less than 240 in seven. All the native peptides were predicted to have less than one minute of half life. Most of the substitutions affected the primary or secondary anchor motifs (leucine in position 2 or valine in position 9 or position 6) but in some cases, a tyrosine was substituted in position 1. This substitution stabilizes the binding of the position 2 anchor residue. The predicted half-lives were also calculated with another online software (Rammensee H G, et al, Immunogenetics 1995;41(4): 178-228) (Table 1).

TABLE 1

HLA A0201 native peptides
and synthetic analogues.

| | | | | |
|---|---|---|---|---|
| | | | | SEQ |
| | | BIMAS | SYFPEITHI | ID |
| Name/type | Sequence | score | score | NO: |

p210-b3a2

| | | | | |
|---|---|---|---|---|
| CMLA2 native | SS [symbol] ALQRPV | 0.003 | 12 | 1 |
| p210F (analogue) | YLKALQRPV | 2.240 | 22 | 2 |
| CMLA3 native | KQSSKALQR | 0.005 | 3 | 3 |
| p210A (analogue) | KQSSKALQV | 24.681 | 13 | 4 |
| p210B (analogue) | KLSSKALQV | 243.432 | 23 | 5 |
| p210Cn native | KALQRPVAS | 0.013 | 10 | 6 |
| p210C (analogue) | KLLQRPVAV | 900.689 | 26 | 7 |
| p210Dn native | TGFKQSSKA | 0.120 | 7 | 8 |
| p210D (analogue) | TLFKQSSKV | 257.342 | 23 | 9 |
| p210E (analogue) | YLFKQSSKV | 1183.775 | 25 | 10 |

p210-b2a2

| | | | | |
|---|---|---|---|---|
| b2a2A native | LTINK [symbol] EAL | 0.247 | 20 | 11 |
| b2a2 A1 (analogue) | LLINKEEAL | 17.795 | 26 | 12 |
| b2a2 A2 (analogue) | LTINKVEAL | 21.996 | 24 | 13 |
| b2a2 A3 (analogue) | YLINKEEAL | 48.151 | 26 | 14 |
| b2a2 A4 (analogue) | YLINKEEAV | 156.770 | 26 | 15 |
| b2a2 A5 (analogue) | YLINKVEAL | 110.747 | 30 | 16 |
| HLA A24 consensus | VYFFLPDHL | | | 21 |
| peptide | | | | |
| positive control | GILGFVFTL | | | 22 |
| influenza matrix | | | | |
| peptide | | | | |

Residues in bold/italics (K in b3a2 and E in b2a2) represent the AA at the fusion breakpoint. Residues underlined represent modifications from the native sequence.

### Example 3

### Mutation of Anchor Residues Increases Binding of HLA-A0201 by BCR-ABL Derived Peptides

### Materials and Experimental Methods

Cell Lines

Cell lines were cultured in RPMI 1640 medium supplemented with 5% FCS, penicillin, streptomycin, 2 mM glutamine and 2-mercaptoethanol at 37° C. in humidified air containing 5% CO₂. T2 is a human cell line lacking TAP1 and TAP2 and therefore unable to present peptides derived from cytosolic proteins.

T2 Assay for Peptide Binding and Stabilization of HLA A0201 Molecules

T2 cells (TAP-, HLA-A0201⁺) were incubated overnight at 37° C. at a concentration of 1×10⁶ cells/ml in FCS-free RPMI medium supplemented with 5 μg/ml human β₂m (Sigma, St Louis, Mo.) in the absence (negative control) or presence of either a positive reference tyrosinase peptide or test peptides at various final concentrations (50, 10, 1, and 0.1 micrograms (μg)/ml). Following a 4-hour incubation with 5 μg/ml brefeldin A (Sigma), T2 cells were labeled for 30 minutes at 4° C. with a saturating concentration of anti-HLA-A2.1 (BB7.2) mAb, then washed twice. Cells were then incubated for 30 minutes, 4° C. with a saturating concentration of FITC-conjugated goat IgG F(ab′)2 anti-mouse Ig (Caltag, San Francisco, Calif.), washed twice, fixed in PBS/1% paraformaldehyde and analyzed using a FACS Calibur® cytofluorometer (Becton Dickinson, Immunocytometry Systems, San Jose, Calif.)

The mean intensity of fluorescence (MIF) observed for each peptide concentration (after dividing by the MIF in the absence of peptide) was used as an indication of peptide binding and expressed as a “fluorescence index.” Stabilization assays were performed similarly. Following initial evaluation of peptide binding at time 0, cells were washed in RPMI complete medium to remove free peptides and incubated in the continuous presence of 0.5 μg/ml brefeldin-A for 2, 4, 6 or 8 hours.

The number of stable peptide-HLA-A2.1 complexes was estimated as described above by immunofluorescence. The half time of complexes is an estimate of the time required for a 50% reduction of the MIF value at time=0.

### Results

To test the computer-generated predicted MHC class 1-binding half-lives of the peptides, the strength of the interaction between the peptides and the HLA-A0201 molecule were directly measured by a binding and stabilization assay that uses the antigen-transporting deficient (TAP2 negative) HLA-A0201 human T2 cells.

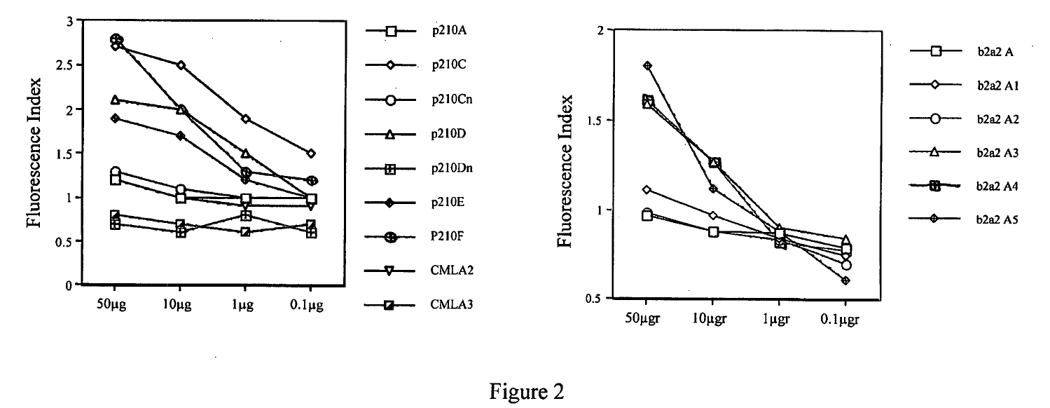

T2 cells lack TAP function and consequently are defective in properly loading class I molecules with antigenic peptides generated in the cytosol The association of exogenously added peptides with thermolabile, empty HLA-A2 molecules stabilizes them and results in an increase in the level of surface HLA-A0201 recognizable by specific mAb such as BB7.2. Seven out eleven peptides designed to have higher binding scores exhibited a relatively high binding affinity for HLA A0201 molecules as measured by the T2 assay (FIG. 2A). A rough correlation between binding scores and binding affinity was established.

Some of these peptides demonstrated the same order of binding affinity as influenza matrix viral antigen, which is among the most potent known antigens for CTL induction. In only four cases was a good correlation between computer-predicted half-life and T2 stabilization not observed.

One of the peptides derived from b3a2, p210C, was mutated from a native peptide that did not have a good prediction score. Nevertheless, the native sequence was able to bind HLA A0201 weakly and at the same level that the previously described CMLA2 peptide. To design p210C, a neutral alanine was substituted for a leucine in position two and a serine was substituted for a valine in position nine. p210C. has a high BIMAS score that correlated with T2 binding assay data (FIG. 2A). p210F is a peptide derived from a sequence that bound weakly in the T2 assay. In this case, the two serines in position one and two were substituted for a tyrosine and a leucine, respectively, with the intent of increasing peptide binding and stabilization to HLA A0201, while retaining the amino-acids for the TCR interaction. The BIMAS prediction showed a 700-fold improvement and binding to T2 cells demonstrated high avidity for HLA A0201 molecules.

Of the peptides derived from b2a2 all were generated from a peptide that was not predicted to have avid binding to HLA A0201. Three new synthetic peptides, b2a2 A3-A5 (Table 1), bound well to HLA A0201 molecules (FIG. 2B). These three peptides had a tyrosine-leucine sequence substitution at position 1 and 2 and also a valine substitution in position 6 or 9, which resulted in increased HLA A0201 binding.

Thus, substitution of anchor residues improved the HLA binding of bcr-abl derived peptides.

### Example 4

### Peptide Analogue Dissociation from HLA A0201

The stability of complexes formed between HLA-A0201 and the b3a2 analogue peptides was assayed with T2 cells. Overnight incubation of T2 cells with saturating amounts of HLA-A0201 binding peptides and human β2 microglobulin resulted in increased surface expression of HLA-A0201 molecules. After peptide removal and addition of brefeldin A to inhibit protein synthesis, the number of HLA-A0201 molecules remaining at the T2 cell surface was determined. The stability of each peptide/HLA-A0201 complex was then normalized relative to that observed for the tyrosinase D peptide or HIV gag peptide (peptides with known high affinity and half life). HLA-A0201 complexes with p210C, p210D, p210E and p210F formed complexes that were stable over 6-8 hours. In contrast, p210A and p210B were less stable, reaching background levels in less than 1 hour of incubation.

These results confirm the results of the previous Example, showing that heteroclitic peptides of the present invention exhibit increased MHC molecule binding.

### Example 5

### Mutated bcr-abl Peptides Stimulate CD8⁺ T Cell Immune Responses Against Mutated and Native Peptides

### Materials and Experimental Methods

Human Dendritic Cell Isolation

PBMC from HLA-A0201 positive healthy donors and chronic myeloid leukemia patients were isolated by Ficoll-density centrifugation. PBMC (DCs) were generated as follows: Monocyte-enriched PBMC fractions were isolated, using a plastic adherence technique, from total PBMC. The plastic-adherent cells were cultured further in RPMI 1640 medium supplemented with 1-5% autologous plasma, 1000 U/mL recombinant human interleukin (IL)-4 (Schering-Plough, N.J.), and 1000 U/mL recombinant human granulocyte-macrophage colony-stimulating factor (GM-CSF) (Immunex, Seattle). On days 2 and 4 of incubation, part of the medium was exchanged for fresh culture medium supplemented with IL-4 and GM-CSF, and culture was continued. On day 6, half of the medium was exchanged for culture medium supplemented with IL-4, GM-CSF, and 10 nanograms (ng)/mL recombinant human tumor necrosis factor (TNF)-alpha (R&D system) and 500 ng/ml of trimeric soluble CD40L (Immunex, Seattle). On day 9, the cells were harvested and used as monocyte-derived DCs for antigen stimulation. The cells generated expressed dendritic cell-associated antigens, such as CD80, CD83, CD86, and HLA class I and class II on their cell surfaces (data not shown).

In Vitro Immunization and Human T Cell Cultures

T lymphocytes were isolated from the same donors by use of negative selection by depletion with an anti-CD11b, anti-CD56 and CD19 MoAb (Miltenyi, Calif.). A total of 1×10⁶ pure T lymphocytes were cultured with 1×10⁵ autologous DC's in RPMI 1640 medium supplemented with 5% heat-inactivated human autologous plasma with bcr-abl synthetic peptides at a concentration of 10 μg/mL and β₂ microglobulin at 2 μg/ml in 24 well plates in the presence of 5-10 ng/mL recombinant human IL-7 (Genzyme) and 0.1 ng/ml of IL-12. After culture for 3 days 20 units (U)/ml of IL-2 was added After 10 days, 1×10⁶ cells were stimulated again by adding 2×10⁵ autologous magnetically isolated CD14⁺ monocytes together with 10 ng/ml of IL-7 and 20 U/ml of IL-2 and 10 μg/mL peptide. In some cases, after culture for another 7 days, the cells were stimulated a third time, in the same manner. After the second or third stimulation, CD8⁺ T cells were magnetically isolated and cytotoxicity and gamma-interferon (IFN) secretion of these cells was determined.

Gamma Interferon ELISPOT

HA-Multiscreen plates (Millipore, Burlington, Mass.) were coated with 100 μl of mouse-anti-human IFN-gamma antibody (10 μg/ml; clone 1-D1K, Mabtech, Sweden) in PBS, incubated overnight at 4° C., washed with PBS to remove unbound antibody and blocked with RPMI/autologous plasma for 1 hour at 37° C. Purified CD8⁺ T cells (more than 95% pure) were plated at a concentration of 1×10⁵/well. T cells were stimulated with 1×10⁴ T2 cells per well pulsed with 10 μg/ml of β₂-microglobulin (Sigma, St. Louis) and either 50 μg/ml of test peptide, positive control influenza matrix peptide GILGFVFTL (Bocchia M et al, Specific human cellular immunity to bcr-abl oncogene-derived peptides. Blood 1996; 87(9): 3587-92), or irrelevant control peptide at a final volume of 100-200 μl/well. Control wells contained T2 cells with or without CD8⁺ cells. Additional controls included medium or CD8⁺ alone plus PBS/5% DMSO diluted according to the concentrations of peptides used for pulsing T2 cells. After incubation for 20 h at 37° C., plates were extensively washed with PBS/0.05% Tween and 100 μl/well biotinylated detection antibody against human IFN-γ (2 μg/ml; clone 7-B6-1. Mabtech, Sweden) were added. Plates were incubated for an additional 2 hours at 37° C. and spot development was performed as described. Spot numbers were automatically determined with the use of a computer-assisted video image analyzer with KS ELISPOT 4.0 software (Carl Zeiss Vision, Germany).

### Results

The next experiments determined the ability of peptides of the present invention to induce activation and proliferation of precursor T cells. Using an optimized T cell-expansion system, with monocyte-derived DC, CD14⁺ cells as APC, and purified CD3⁺ T cells, synthetic b3a2 and b2a2 analogues were evaluated for their ability to stimulate peptide-specific CTLs. Cells from ten healthy HLA A0201 donors and 4 patients with cluonic phase CML were assayed. The peptides used were heteroclitic peptides p210A, p210B, p210C, p210D, and p210E, and CMLA3, p210Cn, p201Dn, and CMLA2, the native sequences corresponding to p210A-B, p210C, p210D, and p210E, respectively (Table 1).

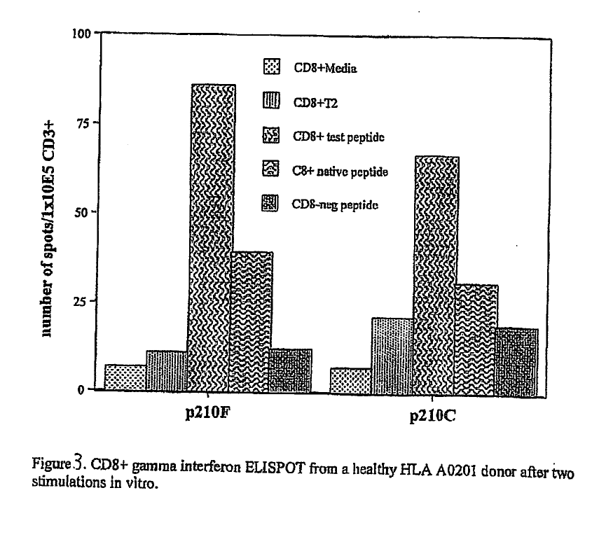

Cells from 5/10 healthy donors responded to immunization, generating T cells that secreted IFN-gamma when challenged with peptide-pulsed T2 cells as targets. p210C and p210F generated the most consistent and significant immune-responses (FIG. 3); p210D and p210E also produced an immune response in some donors tested. Responses were observed after the second or third round of peptide stimulation, either after CD8⁺ isolation or in CD3⁺ T cells not subject to further purification. Spot numbers were consistently higher with peptides that bound with higher affinity to HLA 0201 molecules in the T2 assay. By contrast, no immune response was generated against p210A and p210B, consistent with their reduced affinity for MHC.

In addition, the T cell elicited by p210C and p210F vaccination were able to recognize their respective native sequences (FIG. 3). For example, the peptide CMLA2, the native sequence corresponding to p210F, is a weak MHC binder, and is expressed in the surface of CML blasts.

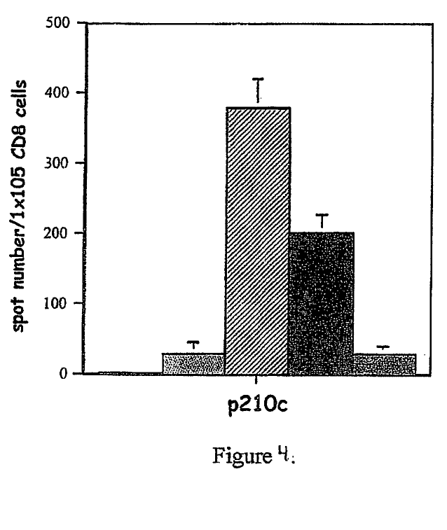

Immune responses to the heteroclitic peptide p21° C. were also observed in two of the CML patients. After two rounds of stimulation with p210C, CD8⁺ cells recognized T2 pulsed with the synthetic peptide with a frequency of nearly 400 spot-forming cells (SCF) per 1×10⁵ cells, and recognized the native peptide on T2 cells with a frequency of 200 SFC per 1×10⁸ (FIG. 4).

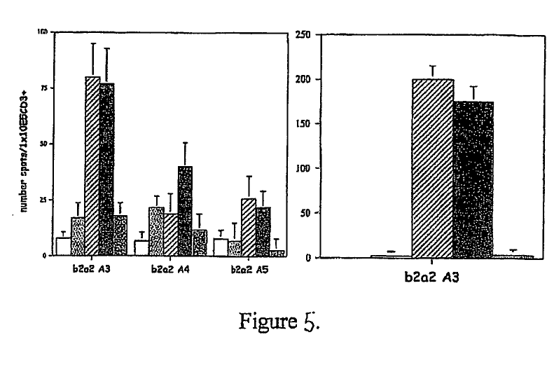

The b2a2-derived peptides A3, A4 and A5 also generated a significant immune response as measured by gamma-IFN secretion by CD3⁺ T cells (FIGS. 5A and 4B), with the response against A3 the most consistent between donors. A3-generated T cells recognized the native sequence as well, despite the fact that the native sequence is a weak HLA binder

Thus, the mutated bcr-abl derived peptides elicited specific T cell immune responses against both the mutated sequences and the original native breakpoint sequences.

### Example 6

### CD8⁺ T Cells Generated by Mutated bcr-abl Peptides are Capable of Cytolysis of Cells Bearing Mutated and Wild-Type bcr-abl Peptides

### Materials and Experimental Methods

Cytotoxicity Assay

The presence of specific CTLs was measured in a standard 4 h-chromium release assay as follows. 4×10⁶ targets were labeled with 300 μCi of Na₂⁵¹CrO₊ (NEN Life Science Products, Inc, Boston, Mass.) for 1 hour at 37° C. After washing, 2×10⁶/ml cells were incubated with or without 10 μg/ml synthetic peptides for 2 hours at 20° C. in presence of 3 μg/ml β₂ microglobulin. After washing by centrifugation, target cells were resuspended in complete media at 5×10⁴ cells per ml and plated in a 96 well U-bottom plate (Becton Dickinson®, NY) at 5×10³ cells per well with effector cells at effector: target ratios (E/T) ranging from 100:1 to 10:1. Plates were incubated for 5 hours at 37° C. in 5% CO₂. Supernatant fluids were harvested and radioactivity was measured in a gamma counter. Percent specific lysis was determined from the following formula: 100×[(experimental release minus spontaneous release)/(maximum release minus spontaneous release)]. Maximum release was determined by lysis of targets in 2.5% Triton X-100.

### Results

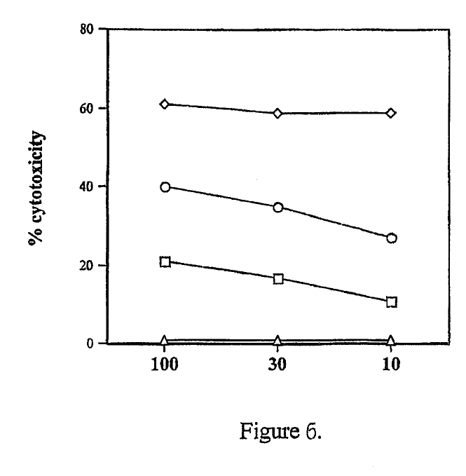

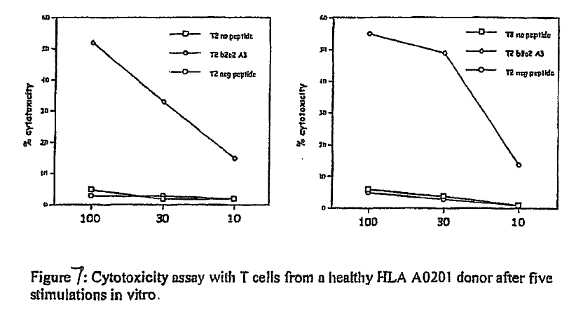

In order to determine whether the in vitro-generated T cells were capable of cytolysis, T cell lines obtained after several stimulations with p210C and b2a2A3 were assayed by chromium-51 release assays using peptide pulsed target cell lines. The cells were able to kill T2 cells pulsed with the heteroclitic peptides. In addition, the cells were able to recognize and kill cells expressing the native peptide from which the heteroclitic peptide was derived (FIGS. 6 and 7). As expected, the cells did not lyse T2 cells without peptide or T2 cells with control peptide, showing the specificity of the assay.

These results confirm the results of the previous Examples, showing that heteroclitic peptides of the present invention exhibit increased immunogenicity relative to the corresponding unmutated (“native”) sequences in both healthy and CML subjects. These results also show that T cells generated with the heteroclitic peptides can recognize MHC molecules bearing the native peptides, even when the native peptide is a weak binder, and can lyse target cells bearing the corresponding peptides.

### Example 7

### GM-CSF and Montanide ISA51 Are Effective Adjuvants for Elicitation of CD8⁺ and CD4⁺ T Cell Immune Response Against Breakpoint Peptides

### Materials and Experimental Methods

CTL Responses

CTL responses were measured by IFN-γ ELISPOT. A mouse CD20 heteroclitic peptide, A3, was used as an antigen (Ag). BALB/c mice (n=5) were injected in the footpads on day 0 and day 14 with peptide alone (20 μg per dose) or mixed with GM-CSF (1 μg per dose) and/or Montanide Isa 51 (50 μL per dose). In the GM-CSF treated groups, GM-CSF was also injected into the footpads two days prior to each immunization, in addition to the GM-CSF mixed with the antigen. CD8⁺ T cells were purified from immunized mice on day 19, and CTL responses was measured by IFN-γ ELISPOT against the syngenic mouse B lymphoma cell line A20, pulsed with A3 or A (native) peptides. Montanide Isa 51 was obtained from Seppic Pharmaceuticals (Fairfield, N.J.).

Antibody Responses

For antibody responses, BALB/c mice (n=5) were injected subcutaneously (SC) with a peptide consisting of 21 AA of human CD20 extracellular domain C terminal (two cysteins are linked with disulfide bound), conjugated to KLH, with or without adjuvant as described above for CTL responses, in this case on days 0, 7 and 21. A week after the last immunization, serum antibody responses against the immunizing peptide and different epitopes of human CD20 were measured by ELISA.

### Results

The abilities of various adjuvants to augment CTL responses to heteroclitic peptides were measured. Mice were injected with peptide mixed with GM-CSF, Montanide Isa 51, or GM-CSF+Montanide ISA 51. CD8⁺ T cells were purified from immunized mice, and CTL responses against cells pulsed with A3 or A (native) peptides were measured The peptides used were derived from CD20; being relatively non-immunogenic, they thus served as a stringent model for induction of anti-peptide immune responses. Strong responses were observed in mice administered peptide+Montanide ISA 51, and the response was further enhanced by 30% with inclusion of GM-CSF in addition to Montanide ISA 51.

Abilities of the adjuvants to augment CD4⁺ T cell responses to peptides were also determined by measuring antibody responses, a surrogate for CD4⁺ T cell responses. Mice were injected with the peptide mixed with either GM-CSF, or Montanide ISA 51, or GM-CSF plus Montanide ISA 51. A week after the last immunization, Ab responses were measured. Strong responses were observed in mice administered GM-CSF alone, Montanide ISA 51 alone, and the two adjuvants in combination.

Thus, GM-CSF and Montanide ISA 51 augment CD4⁺ and CD8⁺ T cell responses. Combining the 2 adjuvants further enhances immune responses.

### Example 8

### b3a2-Derived CML Breakpoint Peptides are Safe and Immunogenic

### Materials and Experimental Methods

Overall Experimental Design

Twelve patients (29-73 years old, also receiving α-interferon or hydroxyurea) participated in the study. Cohorts of 3 patients received 5 subcutaneous injections of escalating doses of peptides mixed with the adjuvant QS-21 over a 10 week period. Four peptides of 9 to 10 AA in length (SSKALQRPV (HLA-A0201 binding; SEQ ID No: 1); KQSSICALQR (HLA-A3 binding; SEQ ID No: 3): ATGFKQSSK (HLA-A11 binding; SEQ ID No: 29); HSATGFKQSSK (HLA-A3/11 binding; SEQ ID No: 30); and GFKQSSKAL (HLA-B8 binding; SEQ ID No: 19) and a 25 AA peptide (IVHSATGFKQSSKALQRPVASDFEP) symmetrically spanning the fusion point were included in the peptide preparation. Peptide-specific T cell proliferative responses, and delayed type hypersensitivity (DTH) responses were assessed at study midpoint and 2 weeks after the last vaccination.

### Results

A phase I dose escalation trial was performed to evaluate the safety and immunogenicity of b3a2-derived CML breakpoint peptides in patients with chronic phase CML. Subjects received escalating doses of peptides mixed with the adjuvant QS-21 in 5 injections over a 10 week period. Three of six patients treated at the 2 highest dose levels of vaccine (500 μg or 1500 μg total peptides) generated peptide-specific T cell proliferative responses, delayed type hypersensitivity (DTH) responses, or both. One patient maintained a response for over 5 months after the final vaccination. Significant adverse effects were not observed.

Thus, bcr-abl derived peptide vaccines are safe and immunogenic in patients with chronic phase CML.

### Example 9

### Mixtures of Native and Synthetic b3a2 and b3a2 Breakpoint-Derived Peptides Elicit Anti-Tumor Immune Responses

### Materials and Experimental Methods

Patients with a b3a2 breakpoint were vaccinated with a preparation of five b3a2 breakpoint-derived native and synthetic peptides plus Montanide ISA 51 and GM-CSF. Patients with a b2a2 breakpoint were vaccinated with b2a2 breakpoint-derived native and synthetic peptides and the same immunologic adjuvant. Patients received the first 5 vaccinations over 8 weeks (see Table 3 below), after receiving a subcutaneous injection of 70 micrograms (mcg) GM-CSF at the vaccine site 2 days and 0 days immediately before injection of peptides Immunologically responding patients received additional monthly vaccinations in the same manner for 10 more months, for a total of 11 vaccinations over approximately 12 months (Table 2). Subjects were observed for 30 minutes after vaccination. Vaccinations were administered subcutaneously, at sites rotated between extremities. Delayed-type hypersensitivity, unprimed ex vivo autologous proliferation (³H-thymidine incorporation), and IFN secretion (ELISPOT assay) were measured before the first vaccination, 2 weeks after the fifth vaccination, and at 2 weeks after the last vaccination In addition, bone marrow aspirates were examined by observation of morphology, cytogenetics, and quantitative PCR for bcr-abl at these time points. HLA typing was performed at study entry if not previously done.

TABLE 2
Timing of vaccinations and assessment of immune responses.

| | Week | | | | | | | | | | | | |
|---|---|---|---|---|---|---|---|---|---|---|---|---|---|
| | Pre | 0 | 2 | 4 | 6 | 8 | 12 | 16 | 20 | 24 | 9 mo | 12 mo | post |
| Vaccination | | X | X | X | X | X | X | X | X | X | X | X | |
| Clinical | | | | | | | | | | | | | |
| follow up | | | | | | | | | | | | | |
| Physical | X | X | X | X | X | X | X | X | X | X | X | X | X |
| exam | | | | | | | | | | | | | |
| CBC | X | X | X | X | X | X | X | X | X | X | X | X | X |
| Chemistries | X | | | | | | X | | | | X | | X |
| Bone | X | | | | | X | | | | | | | X |
| marrow | | | | | | | | | | | | | |
| HLA typing | X | | | | | | | | | | | | |
| Research | | | | | | | | | | | | | |
| assays: | | | | | | | | | | | | | |
| DTH | X | | | | | X | | | | | | | X |
| Proliferation | X | | | | | X | | | | | | | X |
| CD8 | X | | | | | X | | | | | | | X |
| Elispot* | | | | | | | | | | | | | |
| CD4 | X | | | | | X | | | | | | | X |
| Elispot* | | | | | | | | | | | | | |

*In selected patients, in which adequate cells were obtained.

Peptides

Short (9 AA) and long (23-24 AA) peptides were synthesized by F MOC solid phase synthesis and purified by HPLC Purity was assessed by HPLC, and AA sequence was verified by mass spectrometry.

The amino acid sequences of the peptides are set forth in Table 3:

TABLE 3
Sequences of peptides in b2a2 and b3a2 vaccines.

| | | | | |
|---|---|---|---|---|
| | | | Identifier | SEQ |
| | Break- | | from | ID |
| Sequence | point | Type | table 1 | No |

| | | | | |
|---|---|---|---|---|
| VHSIPLTINKEEALQRPV | b2a2 | long, wt | — | 17 |
| ASDFE | | | | |
| YLINKEEAL | b2a2-A2 | synthetic | b2a2 A3 | 14 |
| IVHSATGFKQSSKALQRP | b3a2 | long, wt | — | 18 |
| VASDFE | | | | |
| KQSSKALQR | b3a2-A3 | Wt, A3- | CMLA3 | 3 |
| | | binding | | |
| GFKQSSKAL | b3a2 | Wt, B8- | — | 19 |
| | | binding | | |
| KLLQRPVAV | b3a2 | Synthetic | p210C | 7 |
| YLKALQRPV | b3a2 | Synthetic | p210F | 2 |

Vaccine Specifications

Endotoxin content was demonstrated to be less than 3.0 U/ml by limulus assay. Sterility was confirmed by absence of bacterial and fungal growth on agar plates.

Vaccine Preparation

The two different vaccine preparations were mixed separately with Montanide ISA 51 in a 50:50 ratio and a total volume of 1.50 ml. Peptides were stored at −80° C. and reconstituted in the research pharmacy in PBS (Phosphate-Buffered Saline) in a Nunc® vial by vortexing in a Fisher Scientific vortex machine at highest speed (>3000 rpm) for 12 minutes, then administered to the patient within 2 hours of preparation. Patients were administered subcutaneously 1 ml of the emulsion from a 1-3 ml syringe, using a 25 gauge needle. This vaccine and protocol was approved by the FDA and the IND held by Memorial Sloan Kettering Cancer Center.

GM-CSF

70 mcg GM-CSF was administered subcutaneously in 140 μl at the site of vaccination on day −2 and day 0. GM-CSF was obtained from Berlex Pharmaceuticals (Montville, N.J.) as a sterile, preserved (1.1% benzyl alcohol), injectable 500 mcg/ml solution in a vial. The solution was stored for up to 20 days at 2-8° C. once the vial was punctured, after which the remaining solution in the vial was discarded.

Subject Inclusion Criteria

Adult patients with CML, in major or complete cytogenetic remission but with measurable disease were eligible. Diagnosis of CML was evidenced by a (9;22) translocation or the presence of bcr/abl transcript. Histology, cytogenetics, and bcr/abl transcript analyses were performed at Memorial Hospital of Memorial Sloan-Kettering Cancer Center within four weeks of enrollment.

Major and complete (CCR) cytogenetic remission were defined as (<35% Ph⁺ cells, MCR) and (0% Ph⁺ cells, CCR), respectively. Residual disease was evidenced by detection by qualitative or quantitative reverse transcriptase polymerase chain reaction (RT-PCR) for bcr-abl.

Patients were also required to meet the following criteria:

Presence of the b2a2 or b3a2 breakpoint, as assayed by an approved laboratory. Patients with both breakpoints were assigned to the b3a2 group.

Karnofsky performance status of >70.

Creatinine<2.0 mg/100 ml, bilirubin<2.0 mg/100 ml, LDH<2.0× normal, granulocytes>1,200/mm3, platelets>70,000/mm3, hemoglobin>9 g %.

Age 18 years of age or older.

Ability to give written informed consent.

Subject Exclusion Criteria

Presence of clinically significant heart disease (NYHA class III or IV) or other serious intercurrent illnesses, active uncontrolled infections requiring antibiotics, or active bleeding.

Pregnant or lactating.

Patients requiring corticosteroids or receiving chemotherapy other than Imatinib or interferon were excluded. Patients previous receiving low dose subcutaneous cytarabine were eligible, if the therapy was stopped at least 2 weeks prior to vaccination.

Known immunodeficiency, other than from BMT.

Rapidly accelerating blast counts, “accelerated” or “blastic” CML.

Patients receiving chemotherapy other than Gleevec® (imatinib mesylate), immunotherapy other than interferon, radiation, or donor leukocyte infusion as described below, within 4 weeks prior to vaccination.

Patients taking imatinib or interferon were allowed to enter the study and remain on their entry dose of imatinib or interferon throughout the study.

Patients previously vaccinated with a bcr-abl vaccine were eligible.

Post allogeneic- or autologous bone marrow transplant patients were eligible, if at least six months after the graft.

Concomitant donor leukocyte infusions were allowed within 72 hours after vaccination.

Pre-Treatment Evaluation

Within 2 weeks, of enrollment, subjects received a physical exam and complete blood count (CBC), differential, Na, K, BUN, creatinine, Cl, bilirubin, Ca, PO₄, CO₂, LDH, ALT, pregnancy test if applicable).

Within 4 weeks, when possible, 10 ml bone marrow was collected from subjects and stored for generation of dendritic cells and T cell targets.

Criteria for Removal from Study

A subjects was removed from the study if: 1). S/he received chemotherapy other than Gleevec® or interferon, steroid therapy, or radiation, or failed to comply with the treatment plan. 2). S/he developed progression of disease, requiring systemic treatment, surgery or radiation therapy. 3). S/he requested to discontinue treatment. 4). S/he exhibited toxicity, as determined by observation of a grade 3 Adverse Event, as described in the according to the National Cancer Institute (NCI) Common Toxicity Criteria, version 3.0 (CTCAE3.0).

### Therapeutic Response/Outcome Assessment

Lymphocyte Response

Heparinized peripheral blood (100 to 150 ml) was drawn at the intervals indicated in Table 2, with additional samples drawn 2 weeks after the final vaccination. Peripheral blood lymphocytes (PBLs) were tested for proliferation and γ-IFN release by ELISPOT, in relation to negative controls. When T cell reactivity was observed, additional samples were drawn at 3-6 months after the last vaccination to determine the duration of the response. Laboratory immunogenicity data were assayed at least in triplicate.

Delayed Type Hypersensitivity

DTH against the peptides was determined using standard criteria at indicated intervals, using mumps or candida peptide as a negative control. 10-15 μg of each peptides in PBS were injected intradermally in a volume of 70 μl. Positive responses were defined relative to negative controls, e.g. a two-fold increase in the number of spots by ELISPOT.

Clinical Response

Patients were evaluated by CBC, differential and physical exam at the time of each vaccination and at 2 weeks after the last vaccination. Blood chemistries were performed at 3, 6, and 9 months after study entry and 2 weeks after the last vaccination. Bone marrow evaluations were performed at the intervals indicated in Table 2.

A positive clinical response was defined as conversion from major cytogenetic response to complete cytogenetic response, and for those patients in CCR, by RT-PCR, from molecular positivity to molecular negativity as evidenced by PCR, or by a >1.0 log change by quantitative RT-PCR, provided that the subjects' 2 prior tests were stable. Stability is defined as a less than 0.5 log difference in QRT-PCR or <25% difference in percentage Philadelphia positive by cytogenetic analysis.

### Results

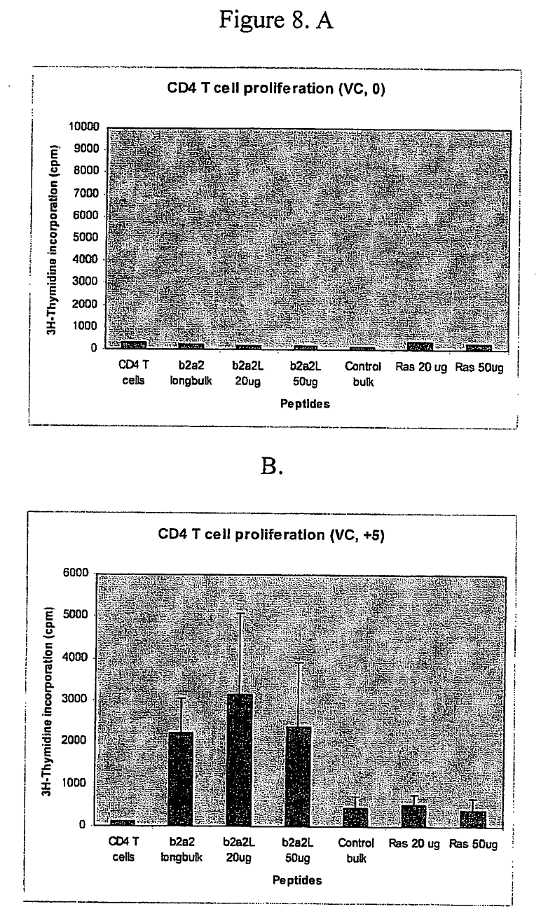

Subjects exhibited measurable bcr-abl-specific immune responses and clinical improvements greater than those observed with either the mutated peptides alone or the unmutated peptides alone. For example, VC, a b2a2 CML patent taking imatinib, received 200 μg b2a2) long peptide in 50% montanide suspension plus 70 μg GM-CSF every 2 weeks. CD4⁺ cells were isolated at time zero (baseline; FIG. 8A) or 2 weeks after the fifth vaccination (B), and stimulated with a mixture of the b2a2 long and short peptides, various negative control peptides (e.g. ras peptide), or no peptide. Antigen-specific CD4⁺ T cell proliferation was observed, as indicated by thymidine incorporation after 20 h stimulation.

Thus, based on the above, a combination of mutated bcr-abl breakpoint peptides and unmutated bcr-abl breakpoint peptides in a vaccine provides enhanced bcr-abl-specific immunogenicity and anti-tumor responses.

1. A bcr-abl vaccine comprising an unmutated bcr-abl peptide, a mutant bcr-abl peptide, and an adjuvant, wherein
a. said unmutated bcr-abl peptide corresponds to a first bcr-abl breakpoint fragment; and
b. said mutant bcr-abl peptide comprises a human leukocyte antigen (HLA) class I-binding peptide, wherein said HLA class I-binding peptide corresponds to a second bcr-abl breakpoint fragment with a mutation in an anchor residue of said second bcr-abl breakpoint fragment.

2. The bcr-abl vaccine of claim 1, wherein said unmutated peptide comprises a human leukocyte antigen (HLA) class II-binding peptide.

3. The bcr-abl vaccine of claim 2, wherein said HLA class II-binding peptide is an HLA-DRB, HLA-DRA, HLA-DQA1, HLA-DQB1, HLA-DPA1, HLA-DPB1, HLA-DMA, HLA-DMB, HLA-DOA, or HLA-DOB binding peptide.

4. The bcr-abl vaccine of claim 1, wherein said second bcr-abl breakpoint fragment is the same as said first bcr-abl breakpoint fragment.

5. The bcr-abl vaccine of claim 1, wherein said second bcr-abl breakpoint fragment is different from said first bcr-abl breakpoint fragment.

6. The bcr-abl vaccine of claim 1, wherein said second bcr-abl breakpoint fragment overlaps with said first bcr-abl breakpoint fragment by at least 7 amino acids.

7. The bcr-abl vaccine of claim 1, wherein said HLA class I-binding peptide is a degradation product of said mutant bcr-abl peptide.

8. The bcr-abl vaccine of claim 1, wherein said mutant bcr-abl peptide consists of said HLA class I-binding peptide.

9. The bcr-abl vaccine of claim 1, wherein said HLA class I-binding peptide is an HLA-A2 binding peptide, an HLA-A3 binding peptide, or an HLA-B8 binding peptide.

10. The bcr-abl vaccine of claim 1, wherein said HLA class I-binding peptide is an HLA-0201 binding peptide.

11. The bcr-abl vaccine of claim 1, wherein administration of said mutant bcr-abl peptide induces an immune response against a cell presenting said second bcr-abl breakpoint fragment.

12. The bcr-abl vaccine of claim 11, wherein said immune response is a heteroclitic immune response.

13. The bcr-abl vaccine of claim 1, further comprising an additional unmutated bcr-abl peptide, wherein said unmutated bcr-abl peptide corresponds to a third bcr-abl breakpoint fragment.

14. The bcr-abl vaccine of claim 13, wherein said additional unmutated peptide comprises a human leukocyte antigen (HLA) class II-binding peptide.

15. The bcr-abl vaccine of claim 14, wherein said HLA class II-binding peptide is an HLA-DRB, HLA-DRA, HLA-DQA1, HLA-DQB1, HLA-DPA1, HLA-DPB1, HLA-DMA, HLA-DMB, HLA-DOA, or HLA-DOB binding peptide.

16. The bcr-abl vaccine of claim 13, further comprising an additional mutant bcr-abl peptide, wherein said additional mutant bcr-abl peptide comprises an additional human leukocyte antigen (HLA) class I-binding peptide, wherein said additional HLA class I-binding peptide corresponds to a fourth bcr-abl breakpoint fragment with a mutation in an anchor residue of said fourth bcr-abl breakpoint fragment.

17. The bcr-abl vaccine of claim 16, wherein said additional HLA class I-binding peptide is a degradation product of said additional bcr-abl mutant peptide.

18. The bcr-abl vaccine of claim 16, wherein said additional mutant bcr-abl peptide consists of said additional HLA class I-binding peptide.

19. The bcr-abl vaccine of claim 16, wherein said additional HLA class I-binding peptide is an HLA-A2 binding peptide, an HLA-A3 binding peptide, or an HLA-B7 binding peptide.

20. The bcr-abl vaccine of claim 16, wherein said additional HLA class I-binding peptide is an HLA-0201 binding peptide.

21. The bcr-abl vaccine of claim 16, wherein said third bcr-abl breakpoint fragment is the same as said fourth bcr-abl breakpoint fragment.

22. The bcr-abl vaccine of claim 16, wherein said third bcr-abl breakpoint fragment is different from said fourth bcr-abl breakpoint fragment.

23. The bcr-abl vaccine of claim 16, wherein said third bcr-abl breakpoint fragment overlaps with said fourth bcr-abl breakpoint fragment by at least 7 amino acids.

24. The bcr-abl vaccine of claim 1, further comprising an additional mutant bcr-abl peptide, wherein said additional mutant bcr-abl peptide comprises an additional human leukocyte antigen (HLA) class I-binding peptide, wherein said additional HLA class I-binding peptide corresponds to a third bcr-abl breakpoint fragment with a mutation in an anchor residue of said third bcr-abl breakpoint fragment.

25. The bcr-abl vaccine of claim 24, wherein said additional HLA class I-binding peptide is a degradation product of said additional bcr-abl mutant peptide.

26. The bcr-abl vaccine of claim 24, wherein said additional mutant bcr-abl peptide consists of said additional HLA class I-binding peptide.

27. The bcr-abl vaccine of claim 24, wherein said additional HLA class I-binding peptide is an HLA-A2 binding peptide, an HLA-A3 binding peptide, or an HLA-B7 binding peptide.

28. The bcr-abl vaccine of claim 24, wherein said additional HLA class I-binding peptide is an HLA-0201 binding peptide.

29. The bcr-abl vaccine of claim 1, wherein said adjuvant is Montanide ISA 51.

30. The bcr-abl vaccine of claim 29, further comprising an additional adjuvant.

31. The bcr-abl vaccine of claim 30, wherein said additional adjuvant is GM-CSF.

32. The bcr-abl vaccine of claim 1, wherein said adjuvant is GM-CSF.

33. The bcr-abl vaccine of claim 32, further comprising an additional adjuvant.

34. The bcr-abl vaccine of claim 1, wherein said adjuvant is a cytokine, a growth factor, a cell population, QS21, Freund's incomplete adjuvant, aluminum phosphate, aluminum hydroxide, BCG, alum, a chemokine, or an interleukin.

35. The bcr-abl vaccine of claim 34, further comprising an additional adjuvant.

36. The bcr-abl vaccine of claim 1, wherein said first bcr-abl breakpoint fragment and said second bcr-abl breakpoint fragment are b3a2 breakpoint fragments.

37. The bcr-abl vaccine of claim 1, wherein said bcr-abl vaccine is a b3a2 bcr-abl vaccine, and wherein said unmutated bcr-abl peptide has an amino acid sequence comprising a sequence selected from IVHSATGFKQSSKALQRPVASDFE (SEQ ID No: 18), KQSSKALQR (SEQ ID No: 3) and GFKQSSKAL (SEQ ID No: 19).

38. The bcr-abl vaccine of claim 1, wherein said bcr-abl vaccine is a b3a2 bcr-abl vaccine, and wherein said unmutated bcr-abl peptide has a sequence selected from IVHSATGFKQSSKALQRPVASDFE (SEQ ID No: 18), KQSSKALQR (SEQ ID No: 3) and GFKQSSKAL (SEQ ID No: 19), or a fragment of SEQ ID No: 18, wherein said fragment is 15-23 amino acids in length.

39. The bcr-abl vaccine of claim 1, wherein said bcr-abl vaccine is a b3a2 bcr-abl vaccine, and wherein said second bcr-abl breakpoint fragment has a sequence selected from SSKALQRPV (SEQ ID No: 1), KQSSKALQR (SEQ ID No: 3), KALQRPVAS (SEQ ID No: 6), or TGFKQSSKA (SEQ ID No: 8).

40. The bcr-abl vaccine of claim 1, wherein said bcr-abl vaccine is a b3a2 bcr-abl vaccine, and wherein said mutated bcr-abl peptide has an amino acid sequence comprising a sequence selected from KLLQRPVAV (SEQ ID No: 7) and YLKALQRPV (SEQ ID No: 2).

41. The bcr-abl vaccine of claim 1, wherein said bcr-abl vaccine is a b3a2 bcr-abl vaccine, and wherein said mutated bcr-abl peptide has a sequence selected from KLLQRPVAV (SEQ ID No: 7) and YLKALQRPV (SEQ ID No: 2).

42. The bcr-abl vaccine of claim 36, further comprising an additional unmutated bcr-abl peptide, wherein said additional unmutated bcr-abl peptide has an amino acid sequence comprising a sequence selected from

| | |
|---|---|
| IVHSATGFKQSSKALQRPVASDFE, | (SEQ ID No: 18) |
| KQSSKALQR | (SEQ ID No: 3) |
| and | |
| GFKQSSKAL. | (SEQ ID No: 19) |

43. The bcr-abl vaccine of claim 36, further comprising an additional unmutated bcr-abl peptide, wherein said additional unmutated bcr-abl peptide has a sequence selected from the sequences IVHSATGFKQSSKALQRPVASDFE (SEQ ID No: 18), KQSSKALQR (SEQ ID No: 3) and GFKQSSKAL (SEQ ID No: 19), or a fragment of SEQ ID No: 18.

44. The bcr-abl vaccine of claim 36, further comprising an additional mutant bcr-abl peptide, wherein said additional mutant bcr-abl peptide comprises an additional human leukocyte antigen (HLA) class I-binding peptide, and wherein said additional mutant bcr-abl peptide comprises a sequence selected from KLLQRPVAV (SEQ ID No: 7) and YLKALQRPV (SEQ ID No: 2).

45. The bcr-abl vaccine of claim 36, further comprising an additional mutant bcr-abl peptide, wherein said additional mutant bcr-abl peptide comprises an additional human leukocyte antigen (HLA) class I-binding peptide, and wherein said additional mutant bcr-abl peptide has a sequence selected from KLLQRPVAV (SEQ ID No: 7) and YLKALQRPV (SEQ ID No: 2).

46. The bcr-abl vaccine of claim 1, wherein said first bcr-abl breakpoint fragment and said second bcr-abl breakpoint fragment are b2a2 fragments.

47. The bcr-abl vaccine of claim 1, wherein said bcr-abl vaccine is a b2a2 bcr-abl vaccine, and wherein said unmutated bcr-abl peptide has a sequence comprising VHSIPLTINKEEALQRPVASDFE (SEQ ID No: 17).

48. The bcr-abl vaccine of claim 1, wherein said bcr-abl vaccine is a b2a2 bcr-abl vaccine, and wherein said unmutated bcr-abl peptide has the sequence VHSIPLTINKEEALQRPVASDFE (SEQ ID No: 17) or a fragment thereof.

49. The bcr-abl vaccine of claim 1, wherein said bcr-abl vaccine is a b2a2 bcr-abl vaccine, and wherein said second bcr-abl breakpoint fragment has the sequence LTINKEEAL (SEQ ID No: 11).

50. The bcr-abl vaccine of claim 1, wherein said bcr-abl vaccine is a b2a2 bcr-abl vaccine, and wherein said mutated bcr-abl peptide has a sequence comprising YLIKEEAL (SEQ ID No: 14).

51. The bcr-abl vaccine of claim 1, wherein said bcr-abl vaccine is a b2a2 bcr-abl vaccine, and wherein said mutated bcr-abl peptide has the sequence YLINKEEAL (SEQ ID No: 14).

52. The bcr-abl vaccine of claim 46, further comprising an additional unmutated bcr-abl peptide.

53. The bcr-abl vaccine of claim 46, further comprising an additional mutated bcr-abl peptide.

54. A method of treating a subject with a bcr-abl-associated cancer, the method comprising administering to said subject the bcr-abl vaccine of claim 1, thereby treating a subject with a bcr-abl-associated cancer.

55. The method of claim 54, wherein said bcr-abl-associated cancer is acute myeloid leukemia, chronic myeloid leukemia, or acute lymphoblastic leukemia.

56. A method of reducing an incidence of a bcr-abl-associated cancer, or its relapse, in a subject, the method comprising administering to said subject the bcr-abl vaccine of claim 1, thereby reducing an incidence of a bcr-abl-associated cancer, or its relapse, in a subject.

57. The method of claim 56, wherein said bcr-abl-associated cancer is acute myeloid leukemia, chronic myeloid leukemia, or acute lymphoblastic leukemia.

58. A method of breaking a T cell tolerance of a subject to a bcr-abl-associated cancer, the method comprising administering to said subject the bcr-abl vaccine of claim 1, thereby breaking a T cell tolerance to a bcr-abl-associated cancer.

59. The method of claim 58, wherein said bcr-abl-associated cancer is acute myeloid leukemia, chronic myeloid leukemia, or acute lymphoblastic leukemia.

60. A method of treating a subject with a cancer associated with a b3a2 bcr-abl chromosomal translocation, the method comprising administering to said subject the bcr-abl vaccine of claim 36, thereby treating a subject with a cancer associated with a b3a2 bcr-abl chromosomal translocation.

61. A method of reducing an incidence of a cancer associated with a b3a2 bcr-abl chromosomal translocation, or its relapse, in a subject, the method comprising administering to said subject the bcr-abl vaccine of claim 36, thereby reducing an incidence of a cancer associated with a b3a2 bcr-abl chromosomal translocation, or its relapse, in a subject.

62. A method of treating a subject with a cancer associated with a b2a2 bcr-abl chromosomal translocation, the method comprising administering to said subject the bcr-abl vaccine of claim 46, thereby treating a subject with a cancer associated with a b2a2 bcr-abl chromosomal translocation.

63. A method of reducing an incidence of a cancer associated with a b2a2 bcr-abl chromosomal translocation, or its relapse, in a subject, the method comprising administering to said subject the bcr-abl vaccine of claim 46, thereby reducing an incidence of a cancer associated with a b2a2 bcr-abl chromosomal translocation, or its relapse, in a subject.

64. A bcr-abl vaccine comprising peptides having the sequences VHSIPLTINKEALQRPVASDFE (SEQ ID No: 17) and YLINKEEAL (SEQ ID No: 14) and an adjuvant.

65. A bcr-abl vaccine comprising peptides having the sequences IVHSATGFKQSSKALQRPVASDFE (SEQ ID No: 18), KQSSKALQR (SEQ ID No: 33), GFKQSSKAL (SEQ ID No: 19), KLLQRPVAV (SEQ ID No: 7), YLKALQRPV (SEQ ID No: 2), and an adjuvant.


SEQUENCE LISTING (94 sequences)

SEQ ID NO: 1 - protein, 9 aa, Homo sapiens
SSKALQRPV

SEQ ID NO: 2 - protein, 9 aa, Homo sapiens
YLKALQRPV

SEQ ID NO: 3 - protein, 9 aa, Homo sapiens
KQSSKALQR

SEQ ID NO: 4 - protein, 9 aa, Homo sapiens
KQSSKALQV

SEQ ID NO: 5 - protein, 9 aa, Homo sapiens
KLSSKALQV

SEQ ID NO: 6 - protein, 9 aa, Homo sapiens
KALQRPVAS

SEQ ID NO: 7 - protein, 9 aa, Homo sapiens
KLLQRPVAV

SEQ ID NO: 8 - protein, 9 aa, Homo sapiens
TGFKQSSKA

SEQ ID NO: 9 - protein, 9 aa, Homo sapiens
TLFKQSSKV

SEQ ID NO: 10 - protein, 9 aa, Homo sapiens
YLFKQSSKV

SEQ ID NO: 11 - protein, 9 aa, Homo sapiens
LTINKEEAL

SEQ ID NO: 12 - protein, 9 aa, Homo sapiens
LLINKEEAL

SEQ ID NO: 13 - protein, 9 aa, Homo sapiens
LTINKVEAL

SEQ ID NO: 14 - protein, 9 aa, Homo sapiens
YLINKEEAL

SEQ ID NO: 15 - protein, 9 aa, Homo sapiens
YLINKEEAV

SEQ ID NO: 16 - protein, 9 aa, Homo sapiens
YLINKVEAL

SEQ ID NO: 17 - protein, 23 aa, Homo sapiens
VHSIPLTINKEEALQRPVASDFE

SEQ ID NO: 18 - protein, 24 aa, Homo sapiens
IVHSATGFKQSSKALQRPVASDFE

SEQ ID NO: 19 - protein, 9 aa, Homo sapiens
GFKQSSKAL

SEQ ID NO: 20 - protein, 17 aa, Homo sapiens
IPLTINKEEALQRPVAS

SEQ ID NO: 21 - protein, 9 aa, Artificial
  consensus peptide
VYFFLPDHL

SEQ ID NO: 22 - protein, 9 aa, Influenza virus
GILGFVFTL

SEQ ID NO: 23 - protein, 17 aa, Homo sapiens
ATGFKQSSKALQRPVAS

SEQ ID NO: 24 - protein, 25 aa, Homo sapiens
IVHSATGFKQSSKALQRPVASDFEP

SEQ ID NO: 25 - protein, 11 aa, Homo sapiens
SKLLQRPVAVD

SEQ ID NO: 26 - protein, 8 aa, Homo sapiens
SKALQRPV

SEQ ID NO: 27 - protein, 11 aa, Homo sapiens
KQSSKALQRPV

SEQ ID NO: 28 - protein, 10 aa, Homo sapiens
QSSKALQRPV

SEQ ID NO: 29 - protein, 9 aa, Homo sapiens
ATGFKQSSK

SEQ ID NO: 30 - protein, 11 aa, Homo sapiens
HSATGFKQSSK

SEQ ID NO: 31 - protein, 22 aa, Homo sapiens
HSIPLTINKEEALQRPVASDFE

SEQ ID NO: 32 - protein, 21 aa, Homo sapiens
HSIPLTINKEEALQRPVASDF

SEQ ID NO: 33 - protein, 22 aa, Homo sapiens
VHSIPLTINKEEALQRPVASDF

SEQ ID NO: 34 - protein, 21 aa, Homo sapiens
SIPLTINKEEALQRPVASDFE

SEQ ID NO: 35 - protein, 21 aa, Homo sapiens
VHSIPLTINKEEALQRPVASD

SEQ ID NO: 36 - protein, 8 aa, Homo sapiens
LINKEEAL

SEQ ID NO: 37 - protein, 8 aa, Homo sapiens
YLINKEEA

SEQ ID NO: 38 - protein, 23 aa, Homo sapiens
VHSATGFKQSSKALQRPVASDFE

SEQ ID NO: 39 - protein, 22 aa, Homo sapiens
VHSATGFKQSSKALQRPVASDF

SEQ ID NO: 40 - protein, 23 aa, Homo sapiens
IVHSATGFKQSSKALQRPVASDF

SEQ ID NO: 41 - protein, 22 aa, Homo sapiens
HSATGFKQSSKALQRPVASDFE

SEQ ID NO: 42 - protein, 22 aa, Homo sapiens
IVHSATGFKQSSKALQRPVASD

SEQ ID NO: 43 - protein, 8 aa, Homo sapiens
QSSKALQR

SEQ ID NO: 44 - protein, 8 aa, Homo sapiens
KQSSKALQ

SEQ ID NO: 45 - protein, 8 aa, Homo sapiens
FKQSSKAL

SEQ ID NO: 46 - protein, 8 aa, Homo sapiens
GFKQSSKA

SEQ ID NO: 47 - protein, 8 aa, Homo sapiens
LLQRPVAV

SEQ ID NO: 48 - protein, 8 aa, Homo sapiens
KLLQRPVA

SEQ ID NO: 49 - protein, 8 aa, Homo sapiens
LKALQRPV

SEQ ID NO: 50 - protein, 8 aa, Homo sapiens
YLKALQRP

SEQ ID NO: 51 - protein, 27 aa, Homo sapiens
KLQTVHSIPLTINKEEALQRPVASDFE

SEQ ID NO: 52 - protein, 26 aa, Homo sapiens
LQTVHSIPLTINKEEALQRPVASDFE

SEQ ID NO: 53 - protein, 25 aa, Homo sapiens
QTVHSIPLTINKEEALQRPVASDFE

SEQ ID NO: 54 - protein, 24 aa, Homo sapiens
TVHSIPLTINKEEALQRPVASDFE

SEQ ID NO: 55 - protein, 10 aa, Homo sapiens
PYLINKEEAL

SEQ ID NO: 56 - protein, 28 aa, Homo sapiens
FLNVIVHSATGFKQSSKALQRPVASDFE

SEQ ID NO: 57 - protein, 27 aa, Homo sapiens
LNVIVHSATGFKQSSKALQRPVASDFE

SEQ ID NO: 58 - protein, 26 aa, Homo sapiens
NVIVHSATGFKQSSKALQRPVASDFE

SEQ ID NO: 59 - protein, 25 aa, Homo sapiens
VIVHSATGFKQSSKALQRPVASDFE

SEQ ID NO: 60 - protein, 10 aa, Homo sapiens
FKQSSKALQR

SEQ ID NO: 61 - protein, 10 aa, Homo sapiens
TGFKQSSKAL

SEQ ID NO: 62 - protein, 10 aa, Homo sapiens
SKLLQRPVAV

SEQ ID NO: 63 - protein, 10 aa, Homo sapiens
QYLKALQRPV

SEQ ID NO: 64 - protein, 27 aa, Homo sapiens
VHSIPLTINKEEALQRPVASDFEPQGL

SEQ ID NO: 65 - protein, 26 aa, Homo sapiens
VHSIPLTINKEEALQRPVASDFEPQG

SEQ ID NO: 66 - protein, 25 aa, Homo sapiens
VHSIPLTINKEEALQRPVASDFEPQ

SEQ ID NO: 67 - protein, 24 aa, Homo sapiens
VHSIPLTINKEEALQRPVASDFEP

SEQ ID NO: 68 - protein, 11 aa, Homo sapiens
YLINKEEALQR

SEQ ID NO: 69 - protein, 10 aa, Homo sapiens
YLINKEEALQ

SEQ ID NO: 70 - protein, 28 aa, Homo sapiens
IVHSATGFKQSSKALQRPVASDFEPQGL

SEQ ID NO: 71 - protein, 27 aa, Homo sapiens
IVHSATGFKQSSKALQRPVASDFEPQG

SEQ ID NO: 72 - protein, 26 aa, Homo sapiens
IVHSATGFKQSSKALQRPVASDFEPQ

SEQ ID NO: 73 - protein, 11 aa, Homo sapiens
KQSSKALQRPV

SEQ ID NO: 74 - protein, 10 aa, Homo sapiens
KQSSKALQRP

SEQ ID NO: 75 - protein, 11 aa, Homo sapiens
GFKQSSKALQR

SEQ ID NO: 76 - protein, 10 aa, Homo sapiens
GFKQSSKALQ

SEQ ID NO: 77 - protein, 11 aa, Homo sapiens
KLLQRPVAVDF

SEQ ID NO: 78 - protein, 10 aa, Homo sapiens
KLLQRPVAVD

SEQ ID NO: 79 - protein, 11 aa, Homo sapiens
YLKALQRPVAS

SEQ ID NO: 80 - protein, 10 aa, Homo sapiens
YLKALQRPVA

SEQ ID NO: 81 - protein, 31 aa, Homo sapiens
KLQTVHSIPLTINKEEALQRPVASDFEPQGL

SEQ ID NO: 82 - protein, 28 aa, Homo sapiens
KLQTVHSIPLTINKEEALQRPVASDFEP

SEQ ID NO: 83 - protein, 29 aa, Homo sapiens
KLQTVHSIPLTINKEEALQRPVASDFEPQ

SEQ ID NO: 84 - protein, 30 aa, Homo sapiens
KLQTVHSIPLTINKEEALQRPVASDFEPQG

SEQ ID NO: 85 - protein, 28 aa, Homo sapiens
TVHSIPLTINKEEALQRPVASDFEPQGL

SEQ ID NO: 86 - protein, 29 aa, Homo sapiens
QTVHSIPLTINKEEALQRPVASDFEPQGL

SEQ ID NO: 87 - protein, 30 aa, Homo sapiens
LQTVHSIPLTINKEEALQRPVASDFEPQGL

SEQ ID NO: 88 - protein, 32 aa, Homo sapiens
FLNVIVHSATGFKQSSKALQRPVASDFEPQGL

SEQ ID NO: 89 - protein, 29 aa, Homo sapiens
FLNVIVHSATGFKQSSKALQRPVASDFEP

SEQ ID NO: 90 - protein, 30 aa, Homo sapiens
FLNVIVHSATGFKQSSKALQRPVASDFEPQ

SEQ ID NO: 91 - protein, 31 aa, Homo sapiens
FLNVIVHSATGFKQSSKALQRPVASDFEPQG

SEQ ID NO: 92 - protein, 29 aa, Homo sapiens
VIVHSATGFKQSSKALQRPVASDFEPQGL

SEQ ID NO: 93 - protein, 30 aa, Homo sapiens
NVIVHSATGFKQSSKALQRPVASDFEPQGL

SEQ ID NO: 94 - protein, 31 aa, Homo sapiens
LNVIVHSATGFKQSSKALQRPVASDFEPQGL